(19) United States
(12) Patent Application Publication
(10) Pub. No.: US 2023/0320707 A1
(43) Pub. Date: Oct. 12, 2023

(54) A SKIN MICROBE SAMPLER AND RELATED METHODS

(71) Applicant: AGENCY FOR SCIENCE, TECHNOLOGY AND RESEARCH, Singapore (SG)

(72) Inventors: Kun Liang, Singapore (SG); Kit Mun Cheryl Leong, Singapore (SG)

(21) Appl. No.: 17/926,714

(22) PCT Filed: Jun. 11, 2021
(86) PCT No.: PCT/SG2021/050341

§ 371 Date: Nov. 21, 2022

(30) Foreign Application Priority Data
Jun. 11, 2020 (SG) 10202005545T

(51) Int. Cl. A61B 10/02; B29C 33/42; B33Y 80/00

(52) U.S. Cl. CPC A61B 10/0233; B33Y 80/00; B29C 33/42

(57) ABSTRACT

The invention relates to a skin microbe sampler comprising: a plurality of solid microneedles extending from a support surface, wherein the microneedles are dimensioned to penetrate the epidermis and/or dermis layer of the skin to obtain microbes residing in the skin. In particular, in one embodiment, the microneedles comprise a cone shape that is substantially devoid of patterning on the surface of said cone shape, in another embodiment, the surfaces of the microneedles comprise an adhesion coating on their surfaces for enhancing adhesion of the microbes on the microneedles. Also provided are a method of sampling microbes residing in a skin, a method of making the microbe sampler, and a mold for making the microbe sampler. In particular, in one embodiment, the method of making the microbe sampler comprises 3D printing the solid microneedles and/or molding the solid microneedles from a negative imprint of the solid microneedles.

### TECHNICAL FIELD

The present disclosure relates broadly to a skin microbe sampler, a method of sampling microbes residing in a skin, a method of making the microbe sampler and a mold for making the microbe sampler.

### BACKGROUND

The tropical climate of Singapore predisposes its residents to a skin microbial overload, resulting in Singapore having the highest incidence of skin disease globally. Many microbial associated skin conditions involve skin barrier disruption (e.g. atopic dermatitis, psoriasis).

Indeed, the skin is colonized by a diverse range of bacteria and fungi, some of which can become potential infectious agents if the skin barrier is compromised. Commensal bacteria can cause severe skin and tissue infection and are a major source of hospital acquired infections and mortality in Intensive Care Units. The emergence of multi-drug resistant drug organisms has steered the development of global antibiotic surveillance programmes. An example is methicillin-resistant Staphylococcus aureus bacteria (MRSA) which is present as a commensal on 30% of the population, causes staphylococcal infections and is a driving factor in atopic dermatitis. Its ability to spread through contact in communities has made its detection and diagnosis paramount in preventing further infection and improving patient care.

Similarly, commensal fungi such as Candida and Dermatophytes may also cause chronic superficial and cutaneous fungal infections such as tinea pedis, Pityriasis versicolor and candidiasis. However, due to limitations in current fungal culture and detection methods, topical antifungals are usually administered without pathological diagnosis.

Absence of accurate detection or misrepresentation of detected species leads to a false understanding of disease aetiology and cumulates in poorer clinical outcomes such as chronic disease. An accurate detection and characterization of skin microbial colonization is thus important in understanding skin health and properly diagnosing invasive bacterial and fungal skin infection.

However, the use of existing sampling tools in the methods of (1) skin swabbing, (2) tape stripping or (3) cup scrubbing that are performed in attempt to diagnose bacterial or fungal infection of the skin, are limited to two-dimensional surface sampling. These conventional methods do not provide sufficiently accurate or representative results that are required for proper disease diagnosis (e.g. high rate of false negatives).

In view of the above, there is a need to address at least ameliorate the above-mentioned problems. In particular, there is a need for better diagnostic tools for skin microbial/microbiome.

### SUMMARY

In one aspect, there is provided a skin microbe sampler comprising: a plurality of solid microneedles extending from a support surface, wherein the microneedles are dimensioned to penetrate the epidermis layer of the skin to obtain microbes residing in the skin.

In one embodiment, the microneedles are configured to obtain microbes residing at a depth that is more than about 20 microns and no more than about 300 microns from the skin surface.

In one embodiment, the microneedles are configured to obtain microbes residing beyond 50% of the depth of the stratum corneum of the epidermis.

In one embodiment, the microneedles comprise a shape that tapers from a base to a tip.

In one embodiment, the microneedles comprise a cone shape that is substantially devoid of patterning on the surface of said cone shape.

In one embodiment, the microneedles have a needle height in the range of from about 1 mm to about 2.2 mm.

In one embodiment, the microneedles have an aspect ratio in the range of about 0.5 to about 4.

In one embodiment, the base comprises a width of from 0.5 mm to 1.5 mm.

In one embodiment, the microneedles are spaced at a distance of from about 0.5 mm to about 2.0 mm from each other on the support surface.

In one embodiment, the microneedles are disposed on the support at an average needle density of about 0.1 needles/mm² to about 0.6 needles/mm².

In one embodiment, the microneedles are arranged in one or more 8×4 array(s).

In one embodiment, the microneedles and the support surface form a unibody.

In one embodiment, the microbe sampler is collapsible to reduce its original surface area for fitting into a chamber having an opening that is smaller than its original surface area.

In one embodiment, the surfaces of the microneedles comprise an adhesion coating on their surfaces for enhancing adhesion of the microbes on the microneedles.

In one embodiment, the microneedles are derived from 3D printing.

In one aspect, there is also provided a method of sampling microbes residing in a skin, the method comprising: contacting the microbe sampler disclosed herein with the skin to allow the microneedles to penetrate the epidermis layer of the skin; allowing microbes residing in the skin to adhere to the microneedles that have penetrated the epidermis layer of the skin; and removing contact of the apparatus from the skin.

In one embodiment, the method of sampling microbes residing in a skin further comprises identifying the presence of microbes on the solid microneedles after removing contact of the microbe sampler from the skin.

In one aspect, there is also provided a method of making the microbe sampler disclosed herein, the method comprising: forming a plurality of solid microneedles that extends from a support surface, wherein the microneedles are dimensioned to penetrate the epidermis layer of the skin to obtain microbes residing in the skin, wherein the forming step comprises 3D printing the solid microneedles and/or molding the solid microneedles from a negative mold of the solid microneedles.

In one embodiment, the method of making the microbe sampler disclosed herein further comprises coating the microneedles with an adhesion coating on their surfaces for enhancing adhesion of the microbes on the microneedles.

In one aspect, there is also provided a mold for making the microbe sampler disclosed herein, the mold comprising: a negative imprint of the solid microneedles.

### Definitions

The term “and/or”, e.g., “X and/or Y” is understood to mean either “X and Y” or “X or Y” and should be taken to provide explicit support for both meanings or for either meaning.

The word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of an apparatus/tool (e.g., a skin microbe sampler), a method of sampling microbes/microbiome residing in the skin, a method of making the apparatus/tool (e.g., microbe sampler), and a mold for making the apparatus/tool (e.g., microbe sampler) are disclosed hereinafter.

There is provided an apparatus/tool (e.g., a skin microbe sampler) for sampling microbes/microbiome residing in the epidermis and/or dermis (including structures like hair follicles, sweat glands etc.), the apparatus/tool comprising, a plurality of microneedles (e.g., solid microneedles) extending from a support surface, wherein the microneedles are capable of penetrating into (or are dimensioned to penetrate) the epidermis layer of the skin to obtain microbes/microbiome residing therein. Advantageously, sampling microbes/microbiome using microneedles may not only address the issue of sampling depth (detection of organisms residing deeper in the epidermis) leading to more representative and accurate data, it may also confer huge advantages for clinicians in time savings (one-time application vs. multiple tape-stripping). Even more advantageously, for the patients, minimally-invasive sub epidermal microneedle sampling will also cause less pain, likely leading to greater patient compliance. Overall, relying on embodiments of the microneedle skin sampling disclosed herein may beneficially provide a much needed standardized depth resolution for addressing the notion of microbial penetration in the skin. Thus, embodiments of the present disclosure challenges existing models of skin sampling which are based on the premise that the skin microbiome is positioned on a uniform two-dimensional plane.

In various embodiments, the apparatus/tool comprises solid microneedles, as opposed to needles with a hollow bore for extracting matter through the bore for example. In various embodiments, the apparatus/tool is different from hollow microneedle sampling as the latter typically involves sampling of interstitial fluids drawn by capillary action. Advantageously, in various embodiments, the apparatus/tool is a sampling tool which can address the three dimensionality of microbial colonization on the skin.

In various embodiments, the microneedles are derived from 3D printing (e.g. the microneedles are 3D printed microneedles). Further, in various embodiments, the support is derived from 3D printing (e.g., the support surface comprises a 3D printed support surface). In various embodiments, being derived from 3D printing may include directly 3D printing the features of the apparatus/tool (e.g., the microneedles and/or the support). Being derived from 3D printing may also include indirectly obtaining the features/apparatus/tool (e.g., the microneedles and/or the support) via one or more 3D printed intermediaries. For example, an intermediary which is 3D printed (e.g. a positive image of the microneedles) may be used as a template to make a mold having a negative imprint/impression of the 3D printed intermediary. The mold may then be used to form the apparatus/tool or its features e.g., via injection molding. Thus, the mold may also be considered as an intermediary for forming the apparatus/tool. In other embodiments, the mold may also be created by directly 3D printing the mold (e.g. directly 3D printing the negative image of the microneedles). In various embodiments, the microneedles comprise 3D printed microneedles and/or the support surface comprises a 3D printed support surface. Advantageously, 3D printing technology allow for rapid and easy prototyping of objects which can be designed using Computer Aided Design (CAD) programs. Thus, 3D printing may overcome the limitations of traditional fabrication methods to create microneedles of complicated geometries. In addition, 3D printing may offer an important advantage in customizability to tailor the microneedle features (needle length, aspect ratio) to suit the patients' skin conditions. Combining a microneedle sampling approach with 3D printing represents a unique approach to microbial diagnostics. Accordingly, various embodiments of the present disclosure fuse expertise in 3D printing, genomic and culturomic detection technologies to provide an unbiased standardized skin sampling tool to enable more accurate and reliable diagnosis of skin conditions.

In various embodiments, the microneedles and the support surface comprise or are formed of the same material. The microneedles and the support surface may also be formed of different material or comprise different material.

In various embodiments, the microneedles comprise or are formed of one of a thermoplastic polymer, thermosetting polymer, photocurable polymer, or combinations thereof. Examples of such polymers include but are not limited to polyamides (PA), polystyrenes (PS), thermoplastic elastomers (TPE), polyaryletherketones (PAEK), polycarbonates (PC), polypropylene (PP), acrylonitrile butadiene styrene (ABS), epoxy resins and the like.

In various embodiments, the microneedles and the support/support surface form part of a unibody. Accordingly, in various embodiments, the microneedles and the support/support surface may be formed as a single body e.g. by continuously 3D printing the microneedles and the support/support surface or through using a single mold of the microneedles and the support/support surface (e.g. injection molding).

In various embodiments, the microneedles are capable of obtaining microbes/microbiomes residing within the stratum corneum of the epidermis. In various embodiments, the microneedles are capable of obtaining microbes/microbiomes residing within the deeper half of the stratum corneum e.g., at more than about 50%, at more than about 55%, at more than about 60%, at more than about 65%, at more than about 70%, at more than about 75%, at more than about 80%, at more than about 85%, at more than about 90%, or at more than about 95% of the depth of the stratum corneum layer.

In some embodiments, the microneedles may be configured to substantially avoid penetrating/breaching layers of the skin that reside below the stratum corneum. In other embodiments, the microneedles may be configured to penetrate/breach layers of the skin that reside below the stratum corneum but are configured to substantially avoid penetrating/breaching at least the deeper half of the dermis layer. The latter embodiments may be advantageous when obtaining microbes residing in or around structures such as hair follicles, sebaceous glands and sweat glands. In various embodiments, the microneedles configured such that they do not or are unable to contact/damage nerves and/or blood vessels beneath the skin. This can advantageously minimise causing pain sensation and bleeding to the subject when the apparatus/device is used.

In various embodiments, the microneedles are configured to obtain microbes residing at a depth that is more than about 20 microns and no more than about 200 microns from the skin surface. In various embodiments, the microneedles are configured to substantially avoid penetrating/breaching a depth of more than about 200 microns, more than about 190 microns, more than about 180 microns, or more than about 170 microns from the skin surface.

Hair follicles and sweat glands can usually go deep and can be found as deep as 1 mm from the skin surface. Therefore, in various embodiments, the microneedles may be configured to further obtain microbes residing at a depth that is more than about 200 microns and no more than about 300 microns. This region may be the region that is in the dermis layer that is close to the epidermis layer and which can be accessed without causing too much pain a subject. In various embodiments, the microneedles can be configured to substantially avoid penetrating/breaching a depth of no more than about 300 microns, no more than about 290 microns, no more than about 280 microns, no more than about 270 microns, no more than about 260 microns, no more than about 250 microns, no more than about 240 microns, no more than about 230 microns, no more than about 220 microns, and no more than about 210 microns.

In various embodiments, the microneedles have a needle height in the range from about 1 mm to about 2 mm for example, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm or about 2.2 mm. In various embodiments, the microneedles may have a needle height of no more than about 1.7 mm, no more than about 1.6 mm, no more than about 1.5 mm, no more than about 1.4 mm, no more than about 1.3 mm, no more than about 1.2 mm, and no more than about 1.1 mm. In various embodiments, the microneedles may have a needle height of no more than about 1.5 mm to minimize discomfort in subjects. In various embodiments, microneedles with a needle height of no more than about 1.5 mm may also be favourable in reducing the risk of deep penetration of the microneedles, particularly in areas with a thinner epidermis. The microneedles of the apparatus/device may have a substantially uniform needle lengths or a mixture of different needle lengths.

In various embodiments, the microneedles comprise a shape that tapers from a base to a tip. In various embodiments, the base is disposed on the support. The base may have a width from about 0.5 mm to about 1.5 mm, for example, about 0.6 mm, about 0.7 mm, about 0.8 mm, about 0.9 mm, about 1.0 mm, about 1.1 mm, about 1.2 mm, about 1.3 mm, about 1.4 mm or about 1.5 mm. In various embodiments, the base may have a radius of 0.5 mm and a diameter of 1 mm. The tip of the microneedle may have a diameter/width of about 10 to 30 μm, i.e., the tip may have a diameter/width of about 10 μm, about 20 μm or about 30 μm.

In various embodiments, the microneedles have an aspect ratio in the range of about 0.5 to about 4, for example, about 1, about 1.1, about 1.2, about 1.3, about 1.4, about 1.5, about 1.6, about 1.7, about 1.8, about 1.9 or about 2, about 2.5, about 2, about 2.5, about 3, about 3.5 or about 4.

In various embodiments, the microneedles comprise a shape that is selected from cone, screw, spike, limpet or the like and combinations thereof. In various embodiments, the conical microneedles have substantially smooth curved surfaces, as well as a flat base and an apex. The microneedles may also be screw-patterned microneedles comprising cone shapes with spiral grooves on the curved surfaces. The microneedles may also be spike-patterned microneedles comprising cone shapes with vertical grooves on the curved surfaces (i.e., the grooves extend from the base to the apex of the microneedles). The microneedles may also be limpet-patterned microneedles comprising cone shapes with surface patterns resembling limpets. In various embodiments, spike-patterned microneedles and limpet-patterned microneedles may substantially have similar/same topographies on cone-shaped bodies and therefore, may be referred to interchangeably in some embodiments. In various embodiments, the microneedles may be cone-shaped microneedles comprising cone shapes that are substantially devoid of patterning (e.g. micropatterning such as grooves, screw-like patterns, limpet-like patterns) on the surface of said cone shapes. Thus, in various embodiments, the transverse cross-sectional areas of the microneedles are substantially circular in shape (c.f. cross-shaped/x-shaped/t-shaped transverse cross-sectional areas of limpet-like microneedles) and/or the longitudinal cross-sectional areas of the microneedles are substantially conical in shape (c.f. branched-shaped longitudinal cross-sectional areas of screw-like microneedles). In various embodiments, an apparatus/tool with cone-shaped microneedles (with substantially smooth curved surfaces) may advantageously provide superior performance in microbe extraction (e.g., bacteria, fungi) and better sampling efficacy as compared to microneedles with other surface patterns (i.e., screw-patterned microneedles, spike-patterned microneedles, and limpet-patterned microneedles).

In various embodiments, the support for the microneedles has a height of from about 0.1 mm to about 1 mm, for example, about 0.1 mm, about 0.2 mm, 0.3 mm, 0.4 mm, 0.5 mm, about 0.6 mm, about 0.7 mm, about 0.8 mm, about 0.9 mm or about 1.0 mm. The support may also be substantially adhesive-free and does not adhere to the skin like conventional tapes.

In various embodiments, the microneedles disposed on the support are spaced at a distance of from about 0.5 mm to about 2 mm, about 0.6 mm to about 1.8 mm, about 0.7 mm to about 1.6 mm, about 0.8 mm to about 1.4 mm, about 0.9 mm to about 1.2 mm or about 1.0 mm to about 1.1 mm from each other.

In various embodiments, the microneedles are disposed on the support at an average needle density of about 0.1 needles/mm² to about 0.6 needles/mm², about 0.1 needles/mm² to about 0.5 needles/mm², about 0.11 needles/mm² to about 0.4 needles/mm², about 0.12 needles/mm² to about 0.3 needles/mm², about 0.13 needles/mm² to about 0.25 needles/mm², about 0.14 needles/mm² to about 0.2 needles/mm², or about 0.10 needles/mm² to about 0.15 needles/mm².

In various embodiments, the microneedles may be arranged in one or more 4×4 array(s), 8×4 array(s), or 16×8 array(s). Thus, the apparatus/device may comprise one or more sets of 4×4 array(s), 8×4 array(s), or 16×8 array(s) of microneedles. In various embodiments, the microneedles arranged in one or more 8×4 array(s) may advantageously provide superior performance in microbe extraction (e.g., bacteria, fungi) and better sampling efficacy as compared to microneedles arranged in one or more 4×4 array(s) or 16×8 array(s) for example.

In various embodiments, the microbes are selected from the group consisting of bacteria, fungi, viruses, protozoa and combinations thereof.

In various embodiments, the microbes are selected from Staphylococcus bacteria species, Malassezia fungi species, Cutibacterium/Propionibacterium bacteria species, Aspergillus fungi species, Streptococcus bacteria species, Pseudomonas bacteria species, Mycobacterium bacteria species or combination thereof. The Staphylococcus bacteria species include but are not limited to Staphylococcus epidermidis and Staphylococcus aureus. The Malassezia fungi species include but are not limited to M. restricta, M. globosa, M. sympodalis, M. slooffiae, and M. furfur. The Cutibacterium/Propionibacterium bacteria species include but are not limited to C. acnes/P. acnes. Other microbes and microbiomes thereof that reside in the skin may be also captured by the apparatus/device.

In various embodiments, the surfaces of the microneedles may be modified to enhance adhesion of microbes thereon. In various embodiments, the microneedles comprise coating (e.g., adhesive coating) on their surfaces to increase/enhance the adhesion of microbes onto the microneedles. The adhesive coating may for example rely on spray adhesives from commercially available sources (e.g., from 3M such as the spray adhesive 3M super 77).

In various embodiments, the apparatus/tool is in the form of a patch. In various embodiments, the apparatus/tool may be collapsible to reduce its original surface area for fitting into a chamber having an opening that is smaller than its original surface area (e.g. in the form of a collapsible patch). In some embodiments, the support surface may be substantially flexible and may be capable of being fitted into a tube with an opening that is smaller than the support surface. In some embodiments, the apparatus/tool may be flexible (e.g. in the form of a flexible patch) and is capable of substantially conforming to the contours of the skin when an external force is applied.

In various embodiments, the apparatus/tool is in the form of a skin microbial sampling tool comprising of microneedle patches (MNPs) with defined needle pattern and lengths manufactured by 3D printing. Advantageously, in contrast to conventional sampling tools which only sample at the skin surface, these MNPs can pick up microbes residing in the deeper layers of the epidermis (strata corneum), which leads to increased sampling efficiency and possibly more representative sampling over existing sampling tools. Thereafter, these commensal microbes can be detected and quantified using molecular detection methods such as real-time quantitative PCR. Even more advantageously, the needle lengths and patterning can be customized to the skin on different parts of the body (face, scalp, hands/feet, abdomen etc.) as well as to each individual to account for differences in gender, age ethnicity, etc.

Various embodiments of the apparatus/tool disclosed herein may advantageously be used for one and/or more of the following various applications: microbial sampling on human skin, microbial sampling on pets' skin, microbial sampling for clinical sampling, and pro or prebiotics delivery.

There is also provided a method of sampling microbes residing in the epidermis of a subject, the method comprising contacting the apparatus/tool (e.g., a skin microbe sampler) provided herein with the skin of the subject to allow the microneedles to penetrate the epidermis layer of the skin. The method further comprises allowing microbes residing in the skin to adhere to the microneedles that have penetrated the epidermis layer of the skin; and removing contact of the apparatus from the skin. The method may be of diagnostic value and thus in various embodiments, the method may be a diagnostic method and the apparatus/device disclosed herein is part of a diagnostic kit. The method may also be a minimally invasive method and likewise, the apparatus or tool may be minimally invasive.

In various embodiments, the contacting step comprises applying a force to penetrate the microneedles into the epidermis layer of the skin to obtain microbes residing therein. In various embodiments, the contacting step comprises rolling, swabbing or applying/sticking the apparatus/tool as a patch onto the skin. The contacting step may comprise applying a force that is substantially perpendicular to the surface of the support (i.e. perpendicular to a plane parallel to the surface of the support) and/or substantially parallel to the direction or longitudinal axes of the microneedles; this is distinguished from conventional swabbing where a substantially horizontal force across the skin is applied. Accordingly, the removing contact step may comprise applying a force that is substantially opposite in direction from the contacting force to remove the apparatus from the skin.

In various embodiments, the penetration of the microneedles into the epidermis layer of the skin does not substantially cause any pain sensation and/or bleeding to the subject.

In various embodiments, the contacting step is carried out for at least about 5 seconds, at least about 6 seconds, at least about 7 seconds, at least about 8 seconds, at least about 9 seconds, at least about 10 seconds, at least about 11 seconds, at least about 12 seconds, at least about 13 seconds, at least about 14 seconds, at least about 15 seconds, at least about 16 seconds, at least about 17 seconds, at least about 18 seconds, at least about 19 seconds, or at least about 20 seconds before removal from the skin.

In various embodiments, the contacting step is carried out a skin or portion thereof that has not been disinfected with alcohol (e.g., ethanol) prior to the contacting step. The alcohol may be a solution having at least about 50%, about 60%, or about 70% alcohol. It has been surprisingly found that if the contacting step is carried out without disinfecting the skin with alcohol, the efficacy of microbe/microbiome pick up by the apparatus/device may be enhanced. Accordingly, in various embodiments, the method disclosed herein is devoid of a step of disinfecting the skin.

In various embodiments, the removing step comprises removing the apparatus/tool as a patch from the skin.

In various embodiments, the method further comprises identifying the presence of microbes on the microneedles after removing contact of the apparatus/tool (e.g., a skin microbe sampler) from the skin of the subject.

In various embodiments, the identifying step comprises: extracting nucleic acid sequences of the microbes obtained from the surface of the microneedles; amplifying the extracted nucleic acid sequences; and determining the identity of the amplified nucleic acid sequence.

In various embodiments, the extracting step comprises immersing the microneedles or samples from the microneedles in a DNA extraction buffer.

In various embodiments, the amplifying step comprises performing PCR/qPCR in the presence of primers on the DNA extraction buffer containing the microbial samples.

In various embodiments, the subject comprises a mammalian subject e.g., a human subject.

An exemplary method of collecting bacteria from skin is described below for illustrative purposes. In one example, the method of collecting bacteria from skin comprises applying a microneedle patch to the skin surface; wherein the microneedle patch is manufactured by 3D printing with a biocompatible resin; wherein the microneedle patch has a cone-shaped topography; wherein the needle length is 1.1-1.7 mm; and wherein the bacteria is collected from the epidermis from a 20-200 μm depth.

There is also provided a method of making the apparatus/tool (e.g., a microbe sampler) provided herein, the method comprising: forming a plurality of solid microneedles that extends from a support surface, wherein the microneedles are dimensioned to penetrate the epidermis layer of the skin to obtain microbes residing in the skin, and wherein the forming step comprises 3D printing the solid microneedles and/or molding the solid microneedles from a negative mold of the solid microneedles. The method may further comprise coating microneedles with an adhesion coating on their surfaces for enhancing adhesion of the microbes on the microneedles.

In various embodiments, the 3D printing the solid microneedles comprises 3D printing a plurality of microneedles on a support surface such that the microneedles extend from the support surface.

In various embodiments, the 3D printing comprises a 3D printing method selected from the group consisting of fused deposition modeling (FDM), stereolithography (SLA), digital light processing (DLP), selective laser sintering (SLS), selective laser melting (SLM), laminated object manufacturing (LOM), and digital beam melting (EBM).

In various embodiments, the method further comprises making a template/mold (negative) from the 3D printed microneedles extending from the support.

In various embodiments, the method further comprises forming the apparatus/tool provided herein from the template/mold (negative), for example via injection molding.

In various embodiments, the method further comprises coating microneedles with an adhesion coating on their surfaces for enhancing adhesion of the microbes on the microneedles.

There is also provided a mold for making the apparatus/tool (e.g., microbe sampler) disclosed herein, the mold comprising: a negative imprint of the solid microneedles. Thus, various embodiments of the present disclosure may include 3D printing the optimal MNP configuration and using that as a template to make a mold (negative) and subsequently manufacture the MNPs by injection molding. Advantageously, in this way, the issue of small batch 3D printing can be circumvented and production costs may be lowered.

### BRIEF DESCRIPTION OF FIGURES

FIG. 1 is an OCT (optical coherence tomography) image showing human skin surface topography produced using the Skintell HD-OCT (high-definition optical coherence tomography) (image courtesy of Dr. Tey Hong Liang, NSC).

FIGS. 2A and 2B show serial tape strip species variation for skin bacteria and fungi. FIG. 2A is a diagram with images of serial tape strips (round tape) from adjacent sides of the nose (i.e. nasal skin) which were cultured on fungal specific agar (mLNA, left column) and bacterial specific agar (Plate count agar, right column). Different species growth was observed in successive tape strips (variable morphologies and phenotypes), suggesting that different layers of the epidermis are colonized by different species of bacteria and fungi (and that different species of fungi and bacteria may be cultured from different layers of nasal skin). FIG. 2B shows a graph showing qPCR quantitation of microbial load using pan fungal and pan bacterial primers (i.e. FIG. 2B shows qPCR quantitation of fungal and bacterial copy number counts across 50 tape strip pressings of nasal skin). FIG. 2B shows that 70-80% of skin surface microbial load can be captured in the first 30 epidermal tape strips. FIGS. 2C, 2D and 2E show data relating to Glabella layer specific qPCR copy number quantitation for common skin microbes from selected samples. FIGS. 2C, 2D and 2E show graphs in particular showing qPCR copy number quantitation from the extracted DNA that was performed alongside the relevant calibrated copy number standard curves for M. restricta, S. epidermidis and P. acnes. * p<0.05.

FIG. 3A is a graph showing that microbial species vary over different layers of the SC (stratum corneum). FIG. 3B is a graph showing quantitative PCR performed on 10×8 consecutive tape strips derived from the nasal sidewall of one healthy volunteer showing the relative detection of bacterial (Staphylococcus epidermidis) and fungal species (Malassezia restricta and Malassezia globose). Staphylococcus aureus was not detected in this individual.

FIGS. 4A and 4B are graphs showing that microbial capture varied across different sampling methods. FIGS. 4A and 4B show percentage recovery of M. furfur from original suspension solution as measured by colony forming units (CFU) for each commercial swab tested, and DNA recovery respectively.

FIGS. 5A, 5B and 5C are computer aided designs (CADs) of microneedle patch prototypes. FIG. 5A shows limpet patterned microneedles, FIG. 5B shows screw patterned microneedles and FIG. 5C shows a high-density design for screw patterned microneedles.

FIGS. 6A, 6B, 6C, 6D, 6E and 6F show images and a CAD design of the early prototype of SLA (Stereolithography)-printed screw patterned microneedle patch in a 4 by 4 needle array of decreasing microneedle length. FIG. 6A is a stereomicroscope image, and FIG. 6B is a CAD design of a microneedle patch with the longest needle measuring 1.7 mm, respectively. FIGS. 6C, 6D, 6E and 6F are microscope images of microneedles showing screw patterning on individual microneedles. The scale bar shown in FIG. 6C (“C”) represents 500 μm.

FIG. 7 shows an image of the prototype of FDM (Fused deposition modelling)-printed microneedle patch.

FIGS. 8A and 8B show photographs of the prototype SLA-printed screw patterned microneedle patch being tested on pig skin to assess efficacy of needle penetration and capacity to capture surface microbials. FIG. 8A shows the prototype microneedle patch being applied on porcine skin (“on application”) and FIG. 8B shows the porcine skin after the prototype microneedle patch is removed (“after application”). FIGS. 8C, 8D, 8E and 8F show microscopic images of porcine skin after microneedle application. The scale bar represents 100 μm.

FIGS. 9A and 9B show graphs indicating the mean fungal counts and mean bacterial counts respectively for two versions of the microneedle prototype that were tested—(1) no adhesive (“non-adhesive”) and (2) with a spray on adhesive (from 3M; 3M super 77) on the needle surface (“adhesive”), alongside a no treatment—lysis buffer only control (“control”). DNA was extracted from 3 surface applications of the prototype microneedle patch and analysed using pan fungal (for FIG. 9A) and pan bacterial (for FIG. 9B) primers. Data shown are mean±S.D.

FIG. 10A shows a top view and FIG. 10B shows a transverse view respectively of a computer-aided design (CAD) of another microneedle patch prototype. FIGS. 100, 10D and 10E show images of MNP prototypes comprising of three topographical designs—cone, screw and spike, with four different needle heights on each patch. FIGS. 10F, 10G and 10H show images of 3D-printed MNPs of various designs and needle heights. The scale bar represents 500 μm.

FIG. 11 is a diagram showing a method of design and manufacture of a 3D-printed trans-epidermal microprojection array (MPA) and a workflow for sampling of human scalp microbiome. The scale bar shown in the first row of image of the 3D-printed MPA represents 1 cm, and the scale bars in the second and third rows of images represent 1 mm.

FIGS. 12A, 12B and 12C each show a set of a photograph (top row) and a microscopic image (bottom row) depicting the 3D-printed trans-epidermal MPAs of various size and density with cone-patterned microprojections. FIG. 12A shows a MPA with has a length A of 11 mm and with a 4×4 array of microprojections (herein referred to as “C_4×4”). FIG. 12B shows a MPA with a length B of 22 mm and with a 8×4 array of microprojections (herein referred to as “C_8×4”). FIG. 12C shows a MPA with a length C of 22 mm and has a 16×8 array of microprojections (herein referred to as “C_16×8”). The scale bars in the photographs represent 1 cm, and the scale bars in the microscopic images represent 1 mm. FIGS. 12D and 12E show graphs comparing the efficacies in picking up M. restricta and S. epidermidis respectively by qPCR copy number quantification. * p<0.05, ** p<0.01, *** p<0.001. FIGS. 12F and 12G each show a set of a photograph (first row) and microscopic images (second and third rows) depicting the MPAs containing microprojections of different topographical patterns. The microscopic images in the third row are an enlarged view of the microprojections shown in the microscopic image in the second row. FIG. 14F shows a MPA with a length D of 22 mm and with a 8×4 array of screw-patterned microprojections (herein referred to as “S_8×4”). FIG. 12G shows a MPA with a length E of 22 mm and with a 8×4 array of limpet-patterned microprojections (herein referred to as “L_8×4”). The scale bar in the photographs represent 1 cm, and the scale bar in the microscopic images represent 1 mm. FIGS. 12H and 12I show graphs comparing the efficacies in picking up M. restricta and S. epidermidis by qPCR copy number quantification. The base (with a length of 22 mm) without any microprojections was used as control. * p<0.05, ** p<0.01. 12J show a graph comparing the depth of penetration (mm) of MPAs of different topographies and densities. ** p<0.01, *** p<0.001.

FIGS. 13A, 13B, and 13C show microscopic images of 3D-printed MPAs with cone-patterned microprojections of various heights, namely, 1.0 mm, 1.2 mm and 1.5 mm in microprojection heights respectively (herein referred to as “C_1.0”, “C_1.2” and “C_1.5” respectively). The scale bars shown in FIGS. 13A, 13B, and 15C represent 1 mm. FIGS. 13D, 13E and 13F show microscopic images of histological sections of porcine skin after MPA insertion. The scale bars shown in FIGS. 13D, 13E and 13F represent 200 μm. FIG. 13G shows a graph of depth of penetration of MPAs of various microprojection heights. * p<0.05.

FIGS. 14A, 14B and 14C show, based on Amplicon sequencing of fungal ITS regions, ITS microbial species-level OTU trees in swab, microneedle and tape strip samples respectively by MetaPhlAn analysis. FIG. 14D is a graph showing the relative abundance of different fungal species across different sampling approaches. FIGS. 14E and 14F are graphs showing the between groups t-test to determine species with significant variation between groups (p value <0.05) at various taxon ranks including phylum, class, order, family, genus, and species. FIGS. 14G and 14H show box plots showing Metastat analysis between groups to determine taxa with significant intra-group variation (* q value <0.05 and ** q value <0.01).

FIGS. 15A and 15B show, based on Amplicon sequencing of fungal ITS regions, box plots showing the Beta Diversity indices between different sampling groups (i.e., swab, microneedle/MPA, tape strip) for Weighted and Unweighted unifrac distances respectively. Wilcoxon signed rank test (* p<0.05 and *** p<0.001). FIG. 15C shows a UPGMA cluster tree based on Weighted and Unweighted Unifrac distances respectively. Clusters which segregate by sampling approach or subject source are indicated accordingly.

FIGS. 16A, 16B and 16C show, based on Amplicon sequencing of bacterial 16S regions, 16S microbial species-level OTU trees in swab, microneedle and tape strip samples by MetaPhlAn analysis. FIG. 16D is a graph showing the relative abundance of different fungal species across different sampling approaches. FIGS. 16E and 16F are graphs showing the between groups t-test to determine species with significant variation between groups (p value <0.05) at various taxon ranks including phylum, class, order, family, genus, and species. FIGS. 16G, 16H and 16I show box plots showing Metastat analysis between groups to determine taxa with significant intra-group variation (* q value <0.05).

FIGS. 17A and 17B show, based on Amplicon sequencing of bacterial 16S regions, box plots showing the Beta Diversity indices between different sampling groups (i.e., swab, microneedle/MPA, tape strip) for Weighted and Unweighted unifrac distances respectively. Wilcoxon signed rank test (*p<0.05). FIG. 17C shows a UPGMA duster tree based on Weighted (top) and Unweighted unifrac distances (bottom). Ousters which segregate by sampling approach or subject source are indicated accordingly.

FIGS. 18A and 18B show graphs comparing the efficacies of MPAs with microprojections of different heights in picking up M. restricta and S. epidermidis by qPCR copy number quantification.

FIGS. 19A and 19B show a perspective view and a top view respectively of a computer-aided design (CAD) sketch of the optimized collapsible trans-epidermal MPA design. FIG. 19C shows a photograph of a non-collapsible MPA design which is too wide to fit into a 2-mL Eppendorf tube. FIG. 19D shows a photograph of the broken halves of a collapsible MPA design which can slide into a 2-mL Eppendorf tube. FIGS. 19E and 19F show a top view and a side view respectively of a 2-mL Eppendorf tube with three collapsible MPA (i.e., six broken halves of the collapsible MPA) being fitted into a 2-mL Eppendorf tube.

FIGS. 20A, 20B, 20C and 20D are graphs showing qPCR copy number quantification from selected samples performed alongside the relevant calibrated copy number standard curves for M. restricta, M. globosa, S. epidermidis and P. acnes respectively. * p<0.05.

FIGS. 21A, 21B, 21C, 21D and 21E are graphs showing OTU copy number comparisons of representative fungal and bacterial species derived from amplicon sequencing with different sampling methods (i.e., swab, MPA and tape strip). FIGS. 21A and 21B show fungal OTU copy numbers for Aspergillus spp. (Ascomycota) and Malassezia spp. (Basidomycota) respectively. FIGS. 21C, 21D and 21E show bacterial OTU copy numbers for Streptococcus spp. (Firmicutes), Pseudomonas spp. (Proteobacteria) and Mycobacterium spp. (Actinobacteria) respectively. ** p<0.01.

FIGS. 22A and 22B are graphs showing the amount of bacteria (S. epidermidis) and fungi (M. restricta) detected respectively (Mean±SD, 1/Cq) by qPCR using the respective diagnostic tools when applied on the pig skin model. Statistical analysis was performed using one way ANOVA, followed by Tukey's post-hoc test. Comparisons were made against the cone microneedle patch, where *p<0.05, **p<0.01 and ****p<0.0001.

FIGS. 23A, 23B, 23C, 23D and 23E are graphs showing OTU copy number comparisons of representative fungal and bacterial species derived from amplicon sequencing with different sampling methods (i.e., swab, MPA and tape strip) for different subjects. FIGS. 23A and 23B show fungal OTU copy numbers for Aspergillus spp. and Malassezia spp. respectively. FIGS. 23C, 23D and 23E show bacterial OTU copy numbers for Streptococcus spp., Pseudomonas spp. and Mycobacterium spp. respectively.

### EXAMPLES

Current skin microbial sampling methods include swabbing, tape stripping or cup scrubbing which generate results that are highly dependent on the skin sampling method applied. Furthermore, the current sampling tools used in these methods sample solely from a uniform two-dimensional skin surface. However, contrary to popular belief, it is the inventors' understanding that microbial colonization (commensal bacteria and fungi) at the stratum corneum (SC) skin barrier is not uniformly two dimensional (i.e. only on one surface of the skin) but distributed across various layers and compartments.

Accordingly, as the currently available sample tools have limited capabilities to sample from deeper layers of the epidermis, it is believed that these tools are not able to achieve the comprehensive sampling depth required to obtain accurate diagnostics of the causative organisms which may reside deeper in the epidermis. In other words, the use of these existing sampling tools, which are incapable of capturing disease causing microbes that reside deeper in the epidermis, may lead to inaccurate (e.g. high rate of false negatives) or less representative results. This will in turn compromise the accuracy of disease diagnosis.

Improved sampling tools and methods could drive better treatment design and support a better understanding of skin diseases by enabling more accurate characterization of the skin microbiome in healthy and diseased skin as well as serving as a platform for diagnosing common microbial skin infections.

Thus, the inventors recognise the existence of a clinical need for reliable assessment of the microbiome in the superficial to deep layer of the SC.

As will be shown in the following Examples, through studies and experiments performed, the inventors have eventually developed an alternative skin microbe/microbiome apparatus/device which aims to address the limitations of the current sampling tools and methods that predominantly focus on the skin surface. Indeed, the development of standardized sampling tools that recognize the presence of microbial colonizers in multiple layers of the skin will enable more accurate and reliable diagnosis.

Example embodiments of the disclosure will be better understood and readily apparent to one of ordinary skill in the art from the following discussions and if applicable, in conjunction with the figures. It should be appreciated that other modifications related to e.g., structural changes may be made without deviating from the scope of the invention. Example embodiments are not necessarily mutually exclusive as some may be combined with one or more embodiments to form new exemplary embodiments. The example embodiments should not be construed as limiting the scope of the disclosure.

### Example 1: Understanding Skin Microbial Colonization

In order to develop a unique and accurate alternative platform for the sampling of skin microbiome that is superior to that of conventional methods, it is imperative to first have a better understanding of skin microbial colonization.

1. Commensal Bacteria and Fungi Reside at Different Levels of the Epidermis

The SC is the upper most layer of the epidermis and forms our first line of microbial defence on the skin. It comprises of corneocytes in layers 10-15 cells thick and 10-20 μm deep which are held together by tight epidermal junctions. Contrary to popular belief, microbial colonization at the SC skin barrier is not uniformly two dimensional but distributed across various layers and compartments.

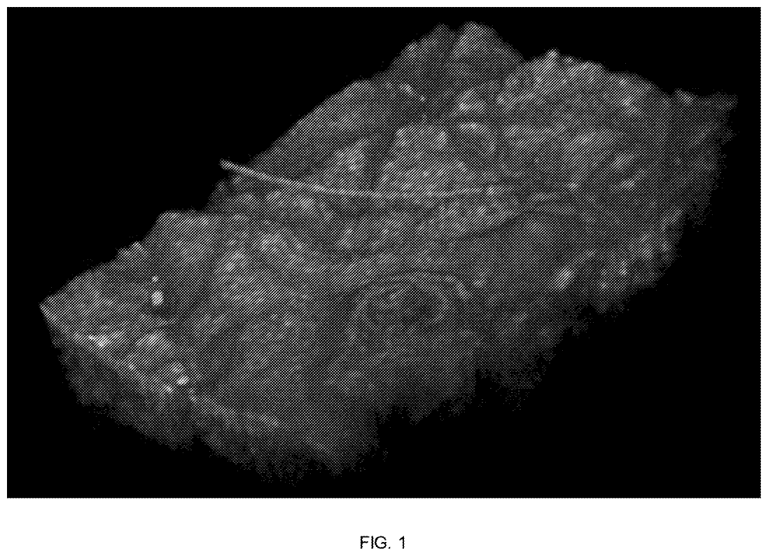

This is nicely illustrated when visualizing OCT images of human skin (FIG. 1) which shows that human skin is highly textured, with numerous striations spanning different depths.

Without being bound by theory, it is believed that bacteria are not homogenously distributed in the SC but vary with superficial and deeper layers of the skin. Using serial tape stripping methods, it has been shown that a majority of bacteria populate the upper layers of the SC and that lower layers are generally sterile.

Species such as Staphylococcus aureus strains associated with atopic dermatitis have been observed to increase in relative abundance in deeper layers of the skin. It has also been demonstrated that that bacterial species composition varies with the thickness of the SC, the presence of hair follicles and other axillary appendages.

Therefore, a reliable assessment of the microbiome in the superficial to deep layer of the SC may eventually allow a more accurate and reliable diagnosis of skin health to be provided.

2. The Skin Microbiome is Three Dimensional and Varies with Skin Layer and Sampling Methods

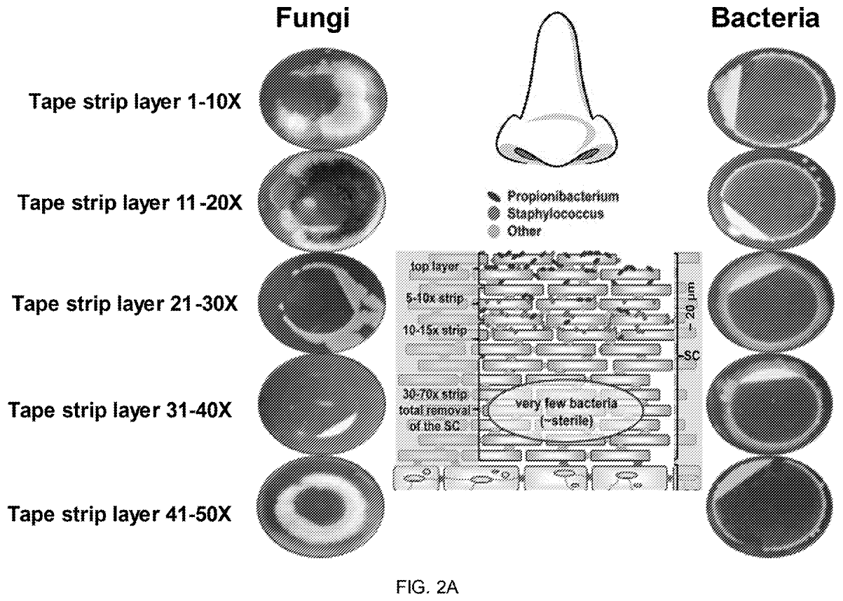

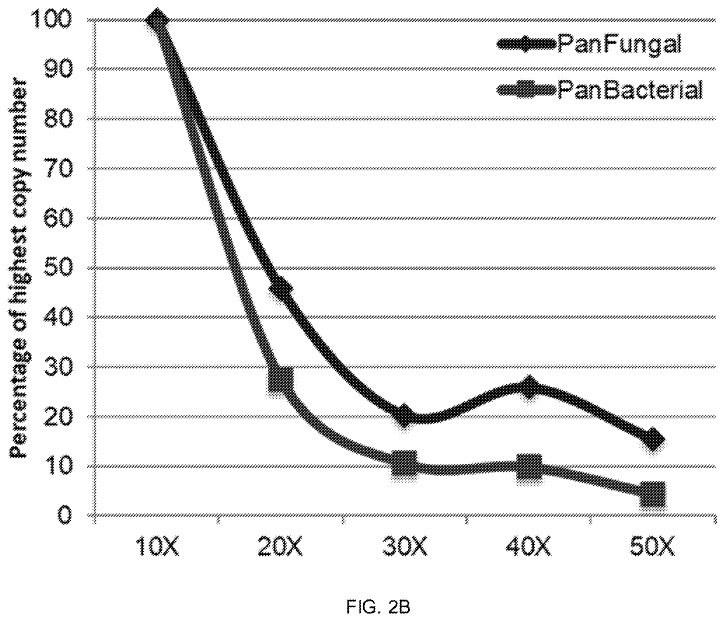

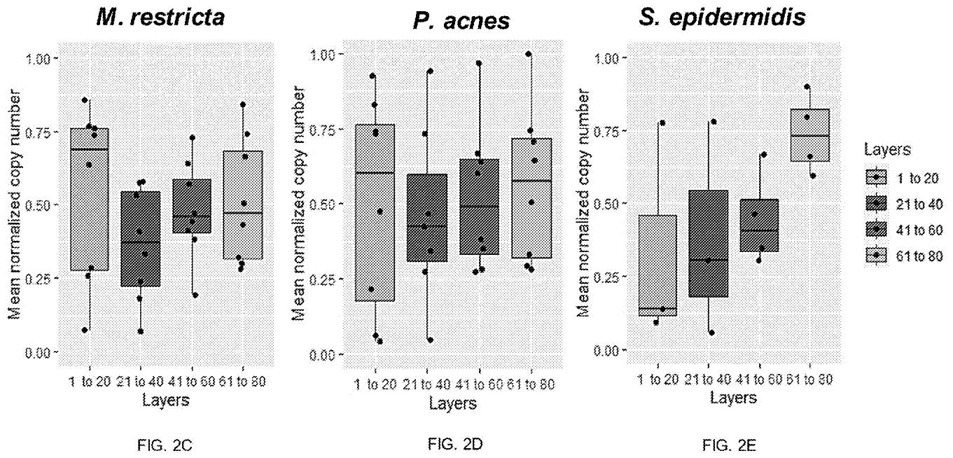

FIG. 2A is a diagram showing the different species of fungi and bacteria that may be cultured from different layers of nasal skin. It was confirmed that the skin microbiome varies in quantity and composition over different layers of the SC using serial tape stripping and serial swabbing methods (FIG. 2A).

FIG. 2B is a graph showing qPCR quantitation of fungal and bacterial copy number counts across 50 tape strip pressings of nasal skin. Commensal bacteria such as S. aureus and S. epidermidis were the most commonly identified on the upper layers and decreased exponentially with each consecutive serial tape strip (FIG. 2B). This is consistent with the understanding that deeper layers of the skin are mostly sterile and have little bacteria. More fungal diversity was observed across the different tape strip layers with filamentous fungi such as Aspergillus and Trichophyton. Tape strip cultures from inner layers of the SC were observed to be dominated by the commensal yeast, Malassezia.

Fungal counts decreased with each successive layer of the skin but did not decrease further after the 30^{th} tape strip.

2A. Glabella Layer Specific qPCR Copy Number Quantitation for Common Skin Microbes from Selected Samples

80× tape strip pressings were obtained from the same region of the Glabella skin in a series of 4×20 pressings to probe microbe density across the layers. The results obtained for M. restricta, S. epidermidis and P. acnes are shown in FIGS. 2C, 2D, 2E respectively. As also shown in these figures, it was confirmed that fungi and bacteria (such as the above) may be found in different layers of the glabella skin as well.

3. Microbial Species Varying Over Different Layers of the SC

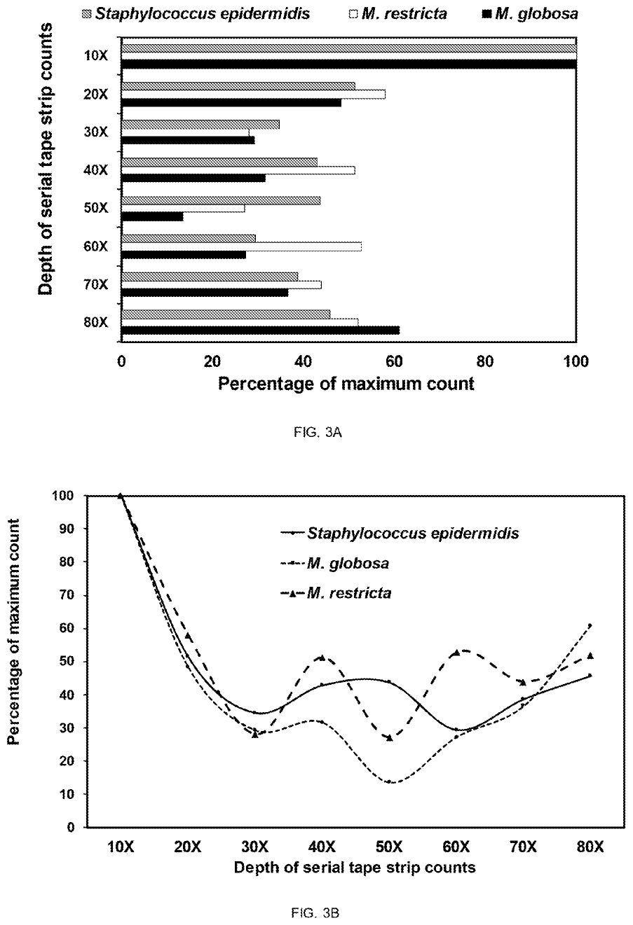

Species specific primers for Staphylococcus epidermidis, Staphylococcus aureus, Malassezia restricta and Malassezia globosa were used to discriminate commensal skin bacterial and fungi. A uniform 70% decrease in bacterial and fungal species counts was observed from the 10th to the 30th tape strip. However, the proportions of different bacterial and fungal species were observed to fluctuate from 10-50% of their maximal counts from a serial strip depth of 30 to 80× (FIGS. 3A and 3B). This supports the hypothesis that microbial species vary over different layers of the SC and suggests that reservoirs of commensal microbes may be present in skin striations which are only accessible at different SC depths.

4. Microbial Capture is Dependent on Sampling Method

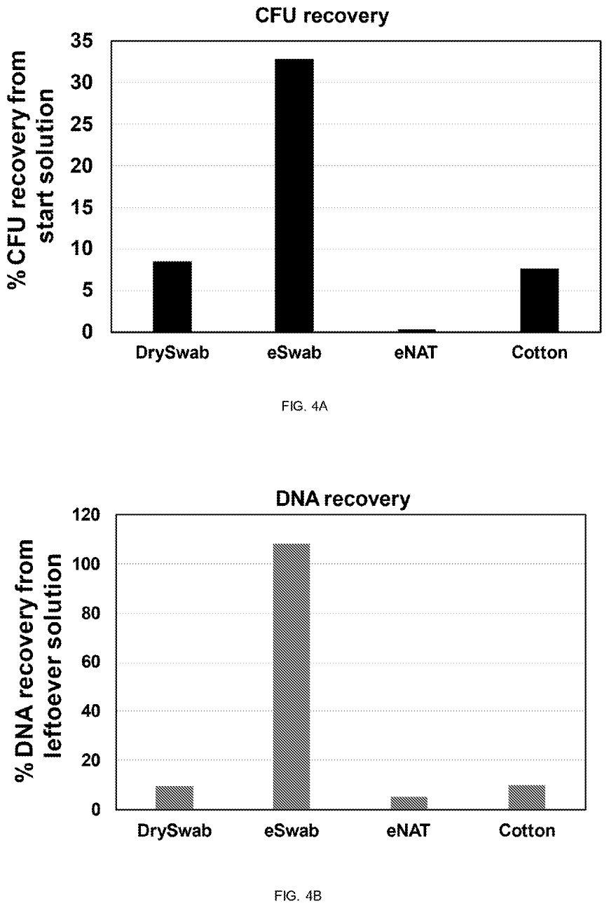

The inventors have established that in addition to culture conditions, the sampling and culture of primary Malassezia fungal isolates is highly dependent on sampling methods. E.g. it is critical that the surface of the skin is not disinfected with 70% ethanol prior to skin swabbing, contrary to what is recommended in some clinical settings. Furthermore, the choice of diagnostic tool (e.g. brand/type of commercial swab) also significantly affected microbial recovery and yield (FIGS. 4A and 4B), suggesting that the results of diagnostic sampling are strongly influenced by the method used.

With a better understanding of the microbial colonization of the skin and how microbial capture may be affected, the inventors then proceeded to devise alternative embodiments of a microneedle-based sampler as will be shown in the Examples below, which will represent a unique and accurate platform for the sampling of skin microbiome. To support the feasibility of this approach, the inventors also have performed some studies with results as illustrated in the Examples below.

### Example 2: 3D-Printed Microneedle Patch (MNP)

Proposed microneedle patch designs Microneedles are three-dimensional (3D) microstructures with microscale projections (e.g., <1000 μm) which are designed to overcome the skin barrier to deliver active molecules in a minimally-invasive manner intradermally by piercing or reversible disruption of the SC.

While some processes can potentially give rise to microneedles with high consistency, they have limited flexibility in terms of the microneedle design. For example, when one type of master template with standard needle lengths and spacing is used as a mold to produce the entire batch of microneedles, it is technically challenging to fabricate microneedles of various geometries such as shape, length, aspect ratio and needle spacing.

Specific microneedle architecture has a direct and critical impact on the quantity of microbial material extracted from the skin. Therefore, it is believed that it is important to fabricate microneedles with customisable geometry and architecture to capture microbial load in the skin layers.

3D printing is one method that has the potential to overcome the limitations of conventional methods because it enables the rapid and facile fabrication of customisable prototypes with complex geometries designed using computer-aided design (CAD) programs. However, even with 3D printing, it may not be straightforward to fabricate microneedles of good resolutions e.g. resolution of printed needles can be poor (the tips were not sharp). Attempting to improve resolution by including a post-printing step of chemical etching may also not be effective as the final printed prototypes may still not well-defined and needles with consistent shapes may not be easily attainable due to the etching step. 3D printing by photopolymerization strategies led to more well-defined microneedles.

In this Example, the inventors have successfully adopted 3D printing for the fabrication of customizable, complex architectures, for the microneedle patch manufacture.

In this Example, microneedle patches (i.e., skin microbe samplers) were designed with the following aims: (1) To penetrate the stratum corneum (SC) for three-dimensional microbial capture; and (2) To maximize microbial capture.

Without being bound by theory, it is believed that the design of the microneedle patch will directly impact the microbial load that is extracted by the needles.

Micro-patterning grooves on a conical surface of 25-30 μm in diameter were envisaged to enhance microbial uptake compared to purely conical ones. To this end, conical needles (i.e., solid microneedles) with patterned surfaces to penetrate the surface of the skin (e.g., penetrate the epidermis layer of the skin) and enable increased microbial capture (i.e., to obtain microbes residing in the skin) via ridges/grooves and large surface area for trapping microbiological material were designed.

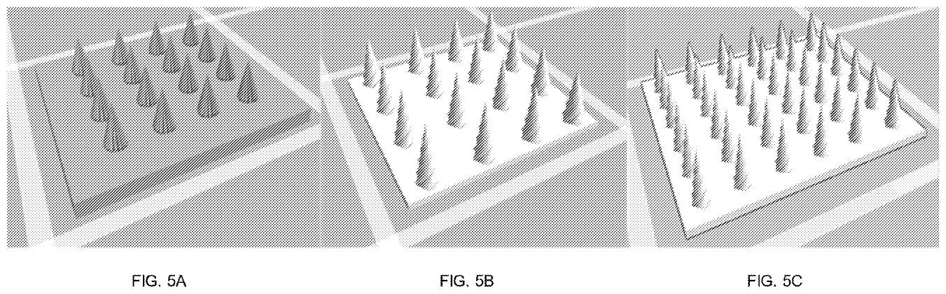

FIGS. 5A and 5B show two key designs that were explored—the limpet pattern, i.e., limpet-patterned microneedles (FIG. 5A) and the screw pattern, i.e., screw-patterned microneedles (FIG. 5B). FIG. 5C shows a high-density design for screw-patterned microneedles.

The tunability and customizability of 3D printing by optimizing the following parameters—needle spacing/density, needle height, thickness of the backing and surface functionalization (e.g., adhesives, in situ hybridization probes) were also be investigated.

3D Printing Feasibility and Prototyping

The following fabrication methods were considered for the approach—(1) Fused deposition modelling (FDM) and (2) Stereolithography (SLA).

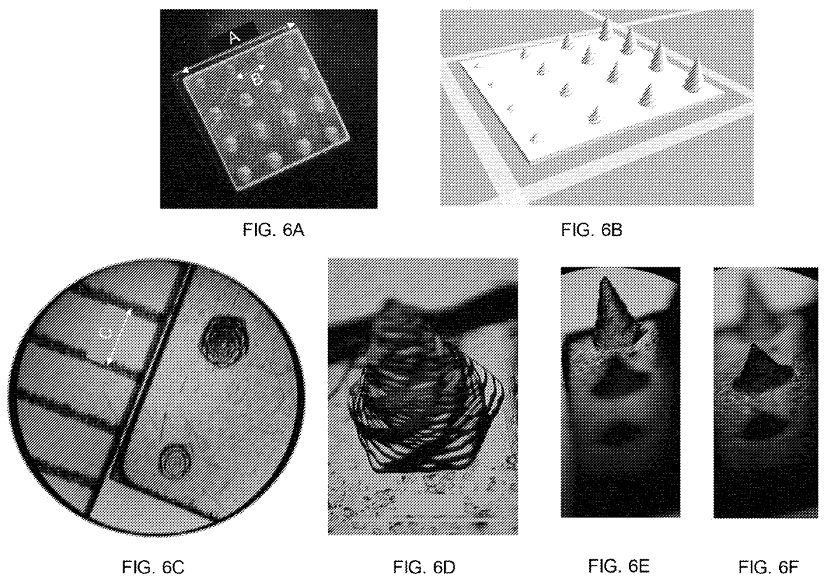

In this Example, a key consideration for 3D microneedle printing is being able to print with high definition and reproducibly at good resolution. To evaluate this across different platforms, CAD software was used to design a test prototype. As shown in FIG. 6A, the prototype designed has a width/length (see distance A in FIG. 6A) of 8 mm and the microneedles are distanced from one another by 1 mm (see distance B in FIG. 6A). The design comprises microneedles endowed with screw patterned contours of various heights (1 mm, 1.3 mm, 1.5 mm and 1.7 mm respectively from the left most column to right most column, as shown in FIG. 6B).

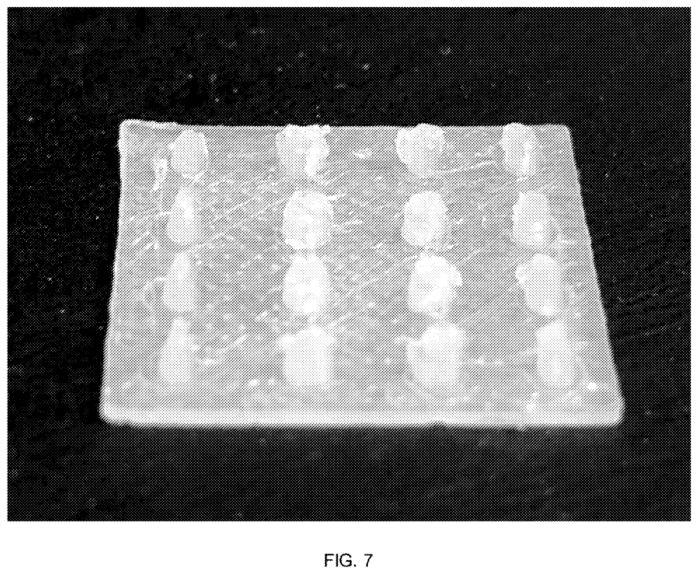

As shown in FIG. 7, early prototyping performed using FDM was not successful as this approach was not able to generate a microneedle patch with high spatial resolution and defined features. The microneedles with the desired shape and form could not be properly printed.

On the other hand, as shown in FIGS. 6C, 6D, 6E and 6F, 3D printing using stereolithography (SLA) was the most successful for the 8×8 mm prototype using the screw patterned needle design.

Under high magnification, a reproducible and defined screw pattern as well as variable needle heights were observed (see FIGS. 6E and 6F for example). The material used for this prototype was PlasCLEAR, which is a proprietary polymer resin provided by the company Asiga.

In this Example, other materials may also be used for 3D printing. For example, the materials may be chosen from the library of biocompatible medical device Class I or II printing materials, i.e., two candidates are Freeprint® temp from DETAX or E-Dent 400 from EnvisionTEC. These materials are FDA-approved for use in dental applications including surgical guides, prosthesis, dentures and aligners.

Testing of Patch Sub-Epidermal Penetration on Porcine Skin

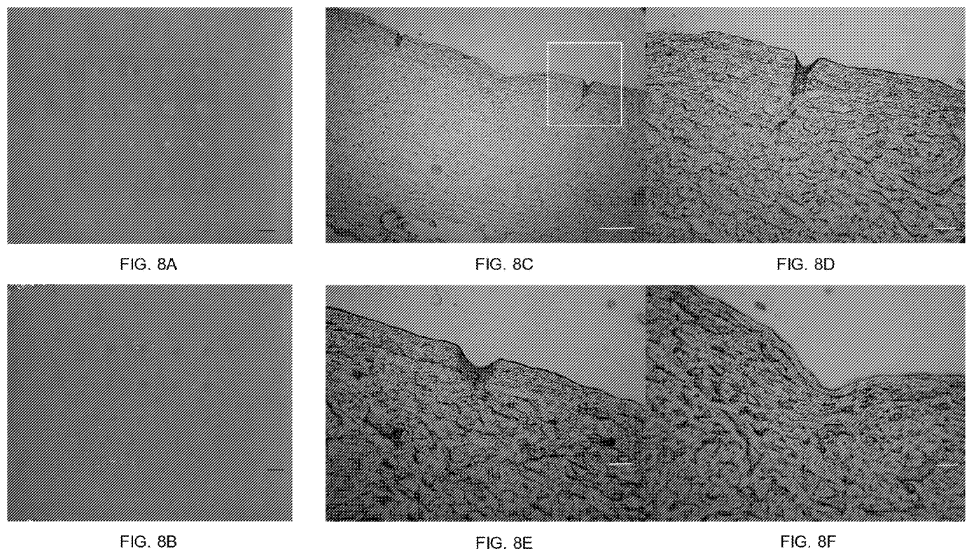

To assess the penetration of SLA printed microneedles patches on the skin, the prototype was applied to strips of porcine skin to assess their depth penetration and ability to pick up microbes from the skin, as shown in FIGS. 8A and 8B.

As shown in FIG. 8B, small needle puncture marks were visible from the surface of the porcine skin after one firm application with a puncture depth of 30-160 μm. FIGS. 8C, 8D, 8E and 8F show images of the punctures/indentations made. FIG. 8D shows an enlarged image of the portion outlined by a rectangle in FIG. 8C. FIG. 8F shows a depth of indentation of 30 μm.

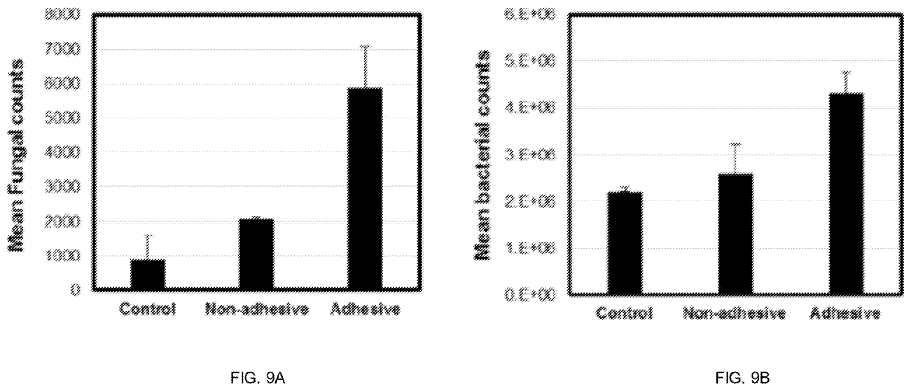

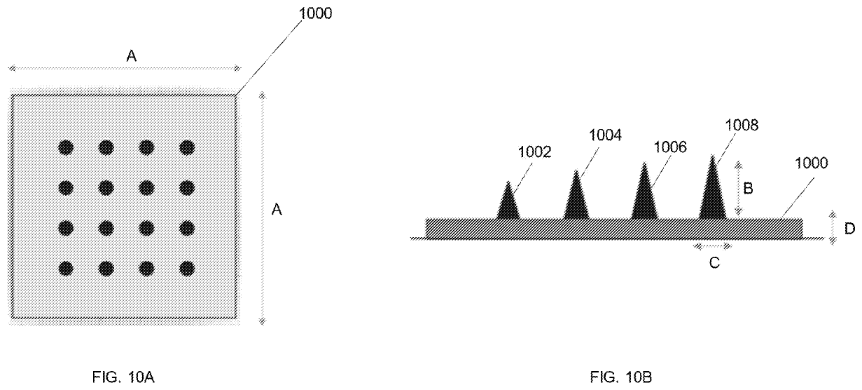

As shown in FIGS. 9A and 9B, the microneedle patch generated higher fungal and bacteria counts compared to control, indicating an enhancement in the sampling quantity.

In this Example, modifications may be made to optimize needle length and patterning to increase microbial capture. Further, species specific genomic tools may be developed for concurrent detection of different microbial species. This can be based on the library of DNA sequences derived from primary microbial isolates from healthy volunteers.

The Example described aims to provide novel microneedle patch designs and novel primer sequence and tools for species-specific microbial detection. Advantageously, embodiments of the present disclosure provide a huge improvement over conventional sampling tools. This is because embodiments of the 3D printed microneedle patch disclosed herein provide more accurate and reliable sampling by enabling 3-dimensional sampling in the deeper layers of the skin as currently available sampling tools tend to only cover the skin surface.

In the Example described, the microneedle patch advantageously is minimally invasive and only for transient use (<1 minute).

### Example 3: 3D-Printed Microneedle Patch (MNP)

In this Example, another example of a 3D-printed microneedle patch (i.e., a skin microbe sampler) is provided.

3D Printing of MNPs with Different Design Parameters

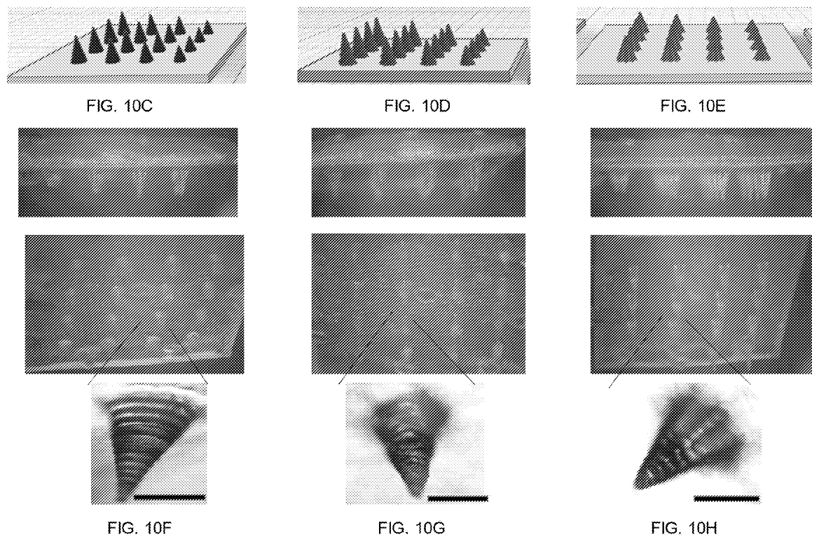

In this Example, MNPs of various designs based on an FDA class 2a biocompatible resin were produced. As shown in FIG. 10A, the microneedle patch 1000 (i.e., a support providing support surface) has a width/length (distance A shown in FIG. 10A) of 11 mm. As shown in FIG. 10B, the microneedle patch 1000 has four columns of solid microneedles 1002, 1004, 1006 and 1008. Microneedles 1002 have a height (i.e., distance measured from its base to its tip; distance B shown in FIG. 10B) of 1 mm, microneedles 1004 have a height of 1.3 mm, microneedles 1006 have a height of 1.5 mm and microneedles 608 have a height of 1.7 mm. The width (or diameter; distance C shown in FIG. 10B) of the microneedles 1002, 1004, 1006 and 1008 are 1 mm. The microneedle patch 1000 has a height/depth (distance D shown in FIG. 10B) of 0.5 mm.

In this Example, two parameters were varied—the needle topography (cone-, screw- or spike-patterned needle, as shown in FIGS. 100, 10D and 10E respectively) and needle height (from 1 to 1.7 mm, as shown in FIG. 10B). Although the different needle heights of MNPs were printed well, the topographical features of the needles were not as well-defined as shown in the CAD drawings (e.g., compare FIG. 100 with FIG. 10F, FIG. 10D with FIG. 10G, and FIG. 10E with FIG. 10H respectively). This was probably due to the limited spatial resolution of the resin (100 μm). Nevertheless, the MNPs were used for microbial uptake evaluation.

In Vitro Testing of MNP Using Sensitive Microbial Molecular Detection Methods

In order to validate the efficacy of the MNP sampling approach, it was necessary to develop de novo sensitive and specific methods for the sensitive and specific detection of skin commensal microbes. This was in the form of genus and species-specific primers for real-time quantitative PCR. This approach was tested and validated. Table 1 provides a summary of primer sets used for the detection of commensal skin microbes as a validation of MNP sampling efficacy.

TABLE 1
Summary of primer sets used for the detection of commensal
skin microbes as a validation of MNP sampling efficacy.
Primer Set

| | Limits of | Limits of | |
|---|---|---|---|
| | Detection | Quantitation | |
| | Colony Forming | Colony Forming | |
| Bacteria | Units (CFU) | Units (CFU) | Source |
| Staphylococcus | 25 CFU | 25 CFU | Published |
| epidermidis | | | |
| Staphylococcus | 5 CFU | 12 CFU | Published |
| aureus | | | |

| | Limits of | Limits of | |
|---|---|---|---|
| | Detection | Quantitation | |
| Malassezia | ITS copy # | ITS copy # | Source |
| M. restricta | 4.01 | 9.68 | In-house |
| M. globosa | 2.77 | 6.63 | Published |
| M. sympodialis | 4.10 | 16.93 | Published |
| M. Slooffiae | 23.44 | 29.51 | In-house design |
| M. furfur | 1.56 | 5.20 | In-house design |

Assessing MNP Efficacy Using Porcine Skin

An assay using genus and species-specific primers for real-time quantitative PCR was used for the quantification of microbial extraction from mock communities of bacteria and fungi on porcine skin. The following were assessed—(1) the effect of topographical features of MNP needles (cone, spike, screw); and (2) the effect of needle lengths of MNPs influencing microbial pick-up on mock skin microbiome communities comprising of S. epidermidis and Malassezia.

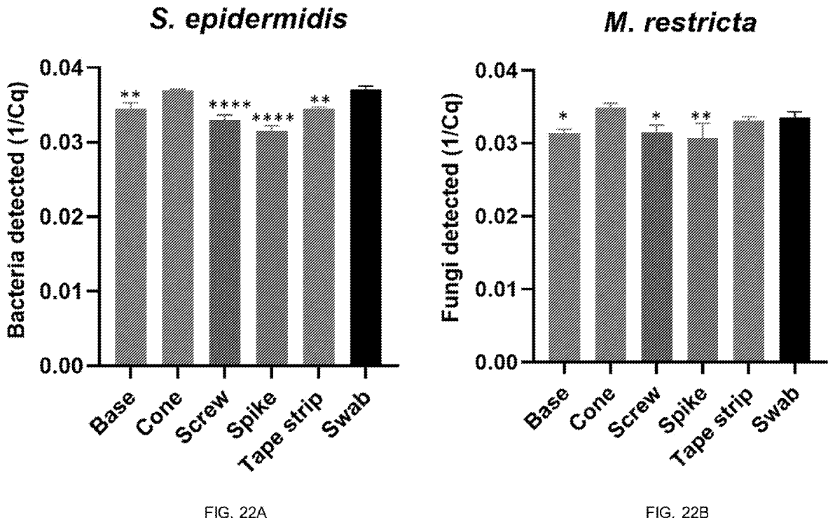

As shown in FIGS. 22A and 22B, the cone-patterned MNP (i.e., MNP with cone-shaped microneedles, with a substantially smooth curved surface) was able to pick up more bacteria and fungi compared to the screw and spike patterns. Most notably, the cone-patterned MNP was also better able to pick up bacteria than one of the conventional sampling methods—tape strip. Swab sampling exhibited the most efficient method of picking up bacteria and fungi in this particular in vitro assay. Nevertheless, it is anticipated that the sampling efficacy of MNPs would be enhanced upon further optimization of the MNP design parameters and functionalization of needle surface (increase needle density, adhesive coating, etc).

Methods

1) 3D Printing of MNPs

MNPs of various needle lengths and topographies were designed using open-source program TinkerCAD. The designs were exported as .stl files and subsequently printed using the Dental LT resin on a Form 2 desktop printer (FormLabs). After printing, the prototypes were cured and rinsed with isopropyl alcohol to remove any unreacted resin.

2) Strains and Growth Conditions

Malassezia reference strains, as listed in Table 2, were obtained from Centraalbureau voor Schimmelcultures (CBS) culture collection. Fresh Malassezia sp. reference strains were maintained in modified Dixon broth (mDixon). Staphylococcus epidermidis was maintained in LB broth, Miller (Millipore, 2768346).

TABLE 2
Reference strains of microbes

| Genus | Species | Reference strain |
|---|---|---|
| Malassezia | globosa | CBS 7966 |
| | restricta | CBS 7866 |
| | sympodialis | CBS 42132 |
| Staphylococcus | epidermidis | ATCC 12228 |

3) Primers and PCR/qPCR Conditions

The list of primers used in the study is listed in Table 3. In quantitative PCR, the reaction mixture was pre-heated to 95.0° C. for 60 seconds, and then 95.0° C. for 15 seconds, and 60.0° C. for 10 seconds for 40 cycles in a two-step amplification process. In PCR, the reaction mixture was pre-heated as per Platinum® Taq DNA Polymerase (ThermoFisher, 10966018) to 94° C. for 5 minutes, then 94.0° C. for 30 seconds, 60.0° C. for seconds and 72.0° C. for 1 minute, repeat for 30 cycles. Lastly, the reaction mix was maintained at 72.0° C. for 7 minutes and cooled to 4° C. indefinitely until further processing was required.

TABLE 3
List of primers

| | | | Expected |
|---|---|---|---|
| | | | product |
| Primers | Forward sequence (5′-3′) | Reverse sequence (5′-3′) | size (bp) |
| VSH-Globosa | GTGAATTGCAGAATTCCGTGAAT | GAGCTTTTTCTAGAGAAGAAAAG | 187 |
| 4F2R- | GTGAATTGCAGAATTCCGTG | AGACACAACCAAACATTCCTC | 322 |
| Restricta | | | |
| VSH- | GTGAATTGCAGAATTCCGTGAAT | TACAATCCCCAGGCAGCAA | 171 |
| Sympodialis | | | |
| SE1-F1R1 | ATCAAAAAGTTGGCGAACCTTTTCA | CAAAAGAGCGTGGAGAAAAGTATCA | 124 |

4) Preparation of Mock Community

Healthy cultures of cells were incubated for 48 to 96 hours prior to conducting the experiments. The OD₆₀₀ was then measured by a ThermoFisher MultiskanSky spectrophotometer on 100 μL of neat S. epidermidis culture. Malassezia sp. cultures—CBS 7966, CBS 7877 and CBS 42132 were diluted 10-fold in Dulbecco PBS (ThermoFisher, 21300025) before measurement. All spectrophotometer measurements were conducted in triplicates. The mock community was then prepared by mixing appropriate volumes of the different cells and diluting respectively with dPBS so that the final OD₆₀₀ of S. epidermidis is 0.1, CBS7966 0.05, CBS7877 0.05 and CBS42132 0.05.

5) Sampling on Pig Skin and Quantitative PCR Analysis

To simulate skin sampling condition, four mL of mock community prepared was smeared onto a sheet of pig (Sus domesticus) skin not exceeding 10 cm in length and width and allowed to settle on the pig skin for 15 minutes. Subsequently, the mock community was decanted and allowed to dry for 30 minutes. The skin was then sampled with the respective methods, i.e., each MNP was pressed on the skin and held firmly for seconds, the tape-strips were applied for 25 consecutive times while the swabs were applied in an orbital manner for 3 cycles. An MNP with only the base (without needles) was used as negative control. After collection, the samples were immersed in 1 mL of DNA extraction buffer with 20 μg/ml of proteinase K (Vivantis, PC0712) and incubated at 56° C. for 18 hours at 500 rpm and heated to 94° C. for 10 minutes. Genomic DNA was extracted with filter sterilised SDS surfactant (Sigma, 71729), glass beads (Sigma, G8772) and phenol-chloroform isoamyl alcohol (Sigma, P3803) and vigorously vortexed and iced. Supernatant with gDNA was pipetted and allowed to precipitate in 100% EtOH (Sigma, E7023) for 24 hours. A quantitative PCR with a standard curve of plasmids at tenfold dilutions was performed against phenol-chloroform isoamyl-extracted DNA using Luna Universal qPCR Master Mix (New England Biolabs, M3003S) using the Agilent AriaMx Real-Time PCR system. The efficacy of sampling was determined by fold-change detection with respect to control.

### Example 4: 3D-Printed Trans-Epidermal Microprojection Array (MPA)

In this Example, a 3D-printed trans-epidermal microprojection array (MPA) (i.e., a skin microbe sampler) is provided.

Method of Manufacturing a 3D-Printed Trans-Epidermal Microprojection Array (MPA) and Workflow for Sampling of Human Scalp Microbe

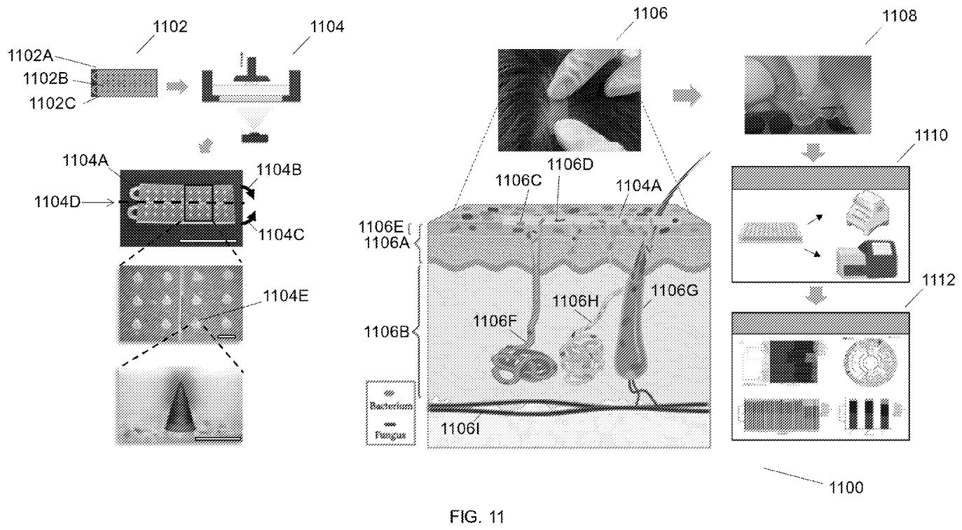

An exemplary method of designing and manufacturing the 3D-printed trans-epidermal microprojection array (MPA) and a workflow for sampling of human scalp microbe are shown in FIG. 11.

In the method 1100, at step 1102, a CAD design of a microneedle sampling tool is designed. The design of the microneedle sampling tool 1102A comprises a plurality of solid microneedles 1102B that extend from a support surface (of a support 1102C), wherein the microneedles 1102B are dimensioned to penetrate the epidermis layer and the dermis layer of the skin to obtain microbes residing in the skin.

At step 1104, based on the CAD design at step 1102, the microneedle sampling tool is 3D-printed (see 3D-printed microneedle sampling tool 1104A). Step 1104 may alternatively involve forming the microneedle sampling tool (including the solid microneedles) from a negative mold thereof.

In this Example, the 3D-printed microneedle sampling tool 1104A is collapsible (i.e., may be separated into half or into two pieces) by folding in the direction of arrows 1104B and 1104C, and along the dotted line 1104D. The 3D-printed microneedle sampling tool 1104A has a plurality of solid microneedles 1104E in a cone shape.

Next, at step 1106, for sampling of human scalp microbiome, the 3D-printed microneedle sampling tool 1104A is placed onto a scalp (skin) of a subject (e.g., a human subject). Contacting the microneedle sampling tool 1104A with the scalp allows the microneedles to penetrate through the epidermis layer 1106A of the skin and to also penetrate (or access) the dermis layer 1106B, so as to obtain microbes (e.g., bacterium 1106C and fungus 1106D) residing in the epidermis layer 1106A and/or the dermis layer 1106B. In particular, the 3D-printed microneedle sampling tool 1104A is capable of obtaining microbes residing in the stratum corneum (SC) 1106E of the epidermis layer 1106A and in the sweat glands 1106F and hair follicles 1106G. In this Example, the 3D-printed microneedle sampling tool 1104A does not interact with the sebaceous glands 1106H and the blood vessels 11061. After allowing contact for a period of time, the microneedle sampling tool 1104A is removed from the scalp (skin). In various embodiments, each MPA may be applied for about five seconds. The MPA may also be applied for about one second, about two seconds, about three seconds, about four seconds, about six seconds, about seven seconds, about eight seconds, about nine seconds or about ten seconds.

At step 1108, the 3D-printed microneedle sampling tool 1104A is separated into two equal pieces and fitted into a (one) 2-mL Eppendorf tube, which is amenable to DNA extraction using minimal buffer, thereby increasing the concentration for downstream processing by real-time PCR and amplicon sequencing.

At step 1110, the obtained sample is processed.

At step 1112, the data obtained is analysed.

Effect of MPA Size, Density and Topography on Microbe Extraction in an Ex Vivo Porcine Skin Model

The effect of MPA size, density and topography on microbe extraction in an ex vivo porcine skin model was evaluated.

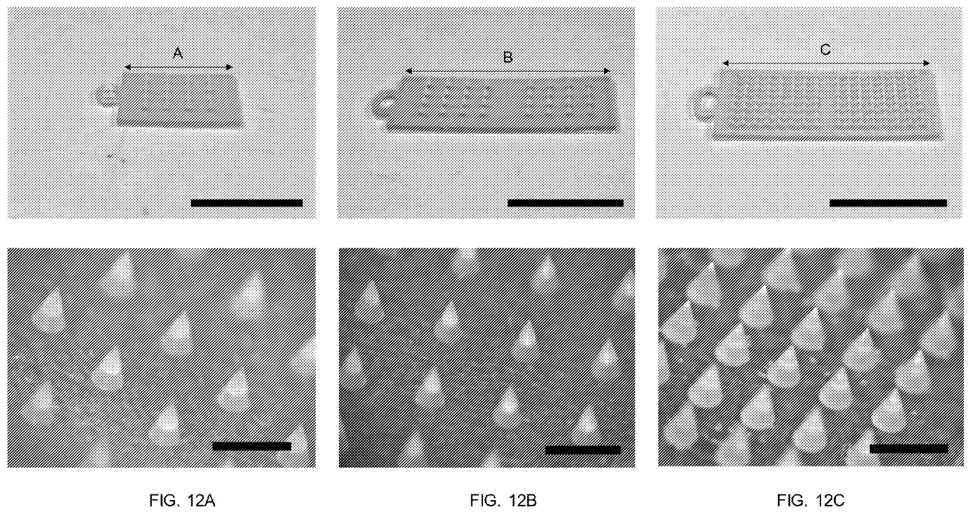

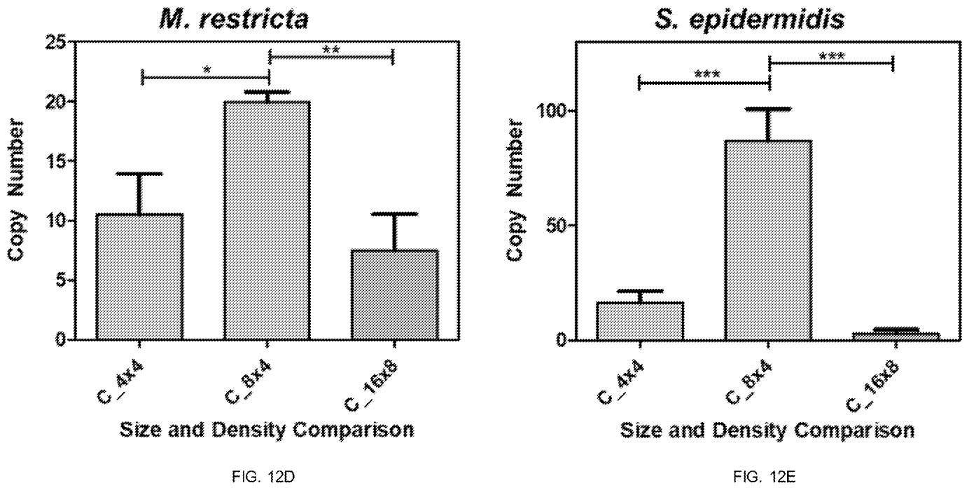

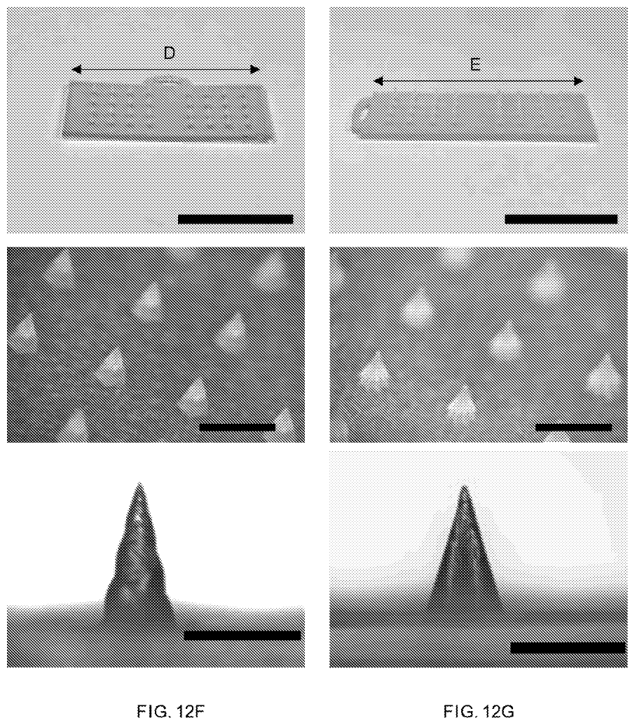

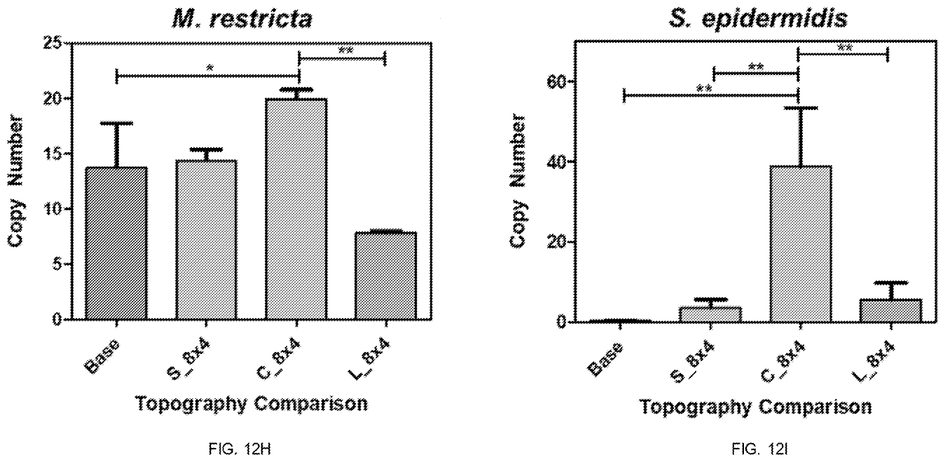

To evaluate the effect of MPA size and density, three different types of MPA were compared, namely, a MPA with has a length A of 11 mm and with a 4×4 array of microprojections (as shown in FIG. 12A; herein referred to as “C_4×4”), a MPA with a length B of 22 mm and with a 8×4 array of microprojections (as shown in FIG. 12B, herein referred to as “C_8×4”), and a MPA with a length C of 22 mm and has a 16×8 array of microprojections (as shown in FIG. 12C; herein referred to as “C_16×8”). Each of these MPAs shown in FIGS. 12A, 12B and 12C have a width of 11 mm.

Amongst the three different types of MPA described above, as shown in FIGS. 12D and 12E, C_8×4 (i.e., MPA with a length B of 22 mm and with a 8×4 array of microprojections) was shown to be the most effective in microbe extraction in the ex vivo porcine skin model.

Next, to evaluate the effect of MPA topography, the following four types of MPA were compared: a base (i.e., a base without any microprojections and with a length of 22 mm) as control, a MPA with a length D of 22 mm and with a 8×4 array of screw-patterned microprojections (as shown in FIG. 12F; herein referred to as “S_8×4”), C_8×4 (i.e., MPA with a length B of 22 mm and with a 8×4 array of microprojections), and a MPA with a length E of 22 mm and with a 8×4 array of limpet-patterned microprojections (as shown in FIG. 12G; herein referred to as “L_8×4”).

Amongst the different types of MPA described above, as shown in FIGS. 12H and 12I, C_8×4 was shown to be the most effective in microbe extraction in the ex vivo porcine skin model.

It was previously hypothesized that patterning could increase pickup/enhance microbial uptake. However, the inventors have surprisingly found that this was not the case, i.e., non-patterned microprojections were more effective. This was an unexpected finding. Without being bound by theory, it is believed that one possible reason may be related to the penetration depth. The patterned surface with grooves seemed to hinder the penetration of the MPA. In other words, depth penetration of the MPAs seemed to be reduced as a result of topographical changes. As shown in FIG. 12J, the cone topography showed deeper penetration depth than the screw, limpet topography MPAs. Also, the MPA with higher density showed lower penetration depth.

Effect of Microprojection Heights on Skin Penetration in Porcine Skin Ex Vivo

The effect of microprojection heights on skin penetration in porcine skin ex vivo was also evaluated.

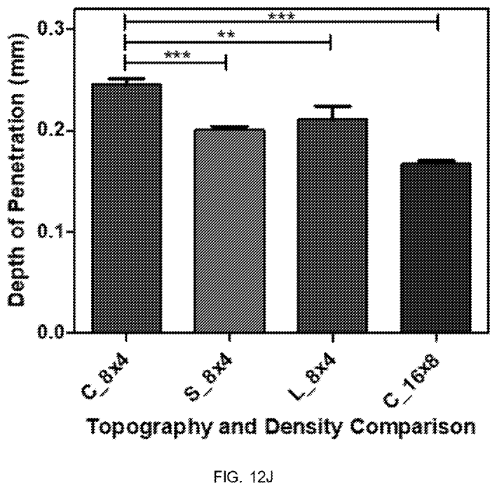

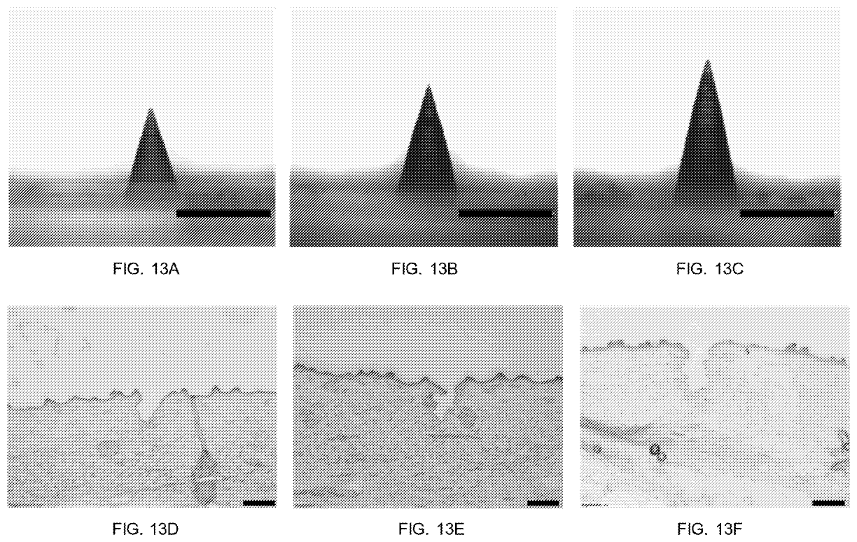

To this end, three different types of 3D-printed MPAs were compared, namely, 3D-printed MPAs with cone-patterned microprojections of 1.0 mm, 1.2 mm and 1.5 mm (as shown in FIGS. 13A, 13B and 13C respectively; herein referred to as “C_1.0”, “C_1.2” and “C_1.5” respectively).

As shown by the graph in FIG. 13G, as well as by the microscopic images shown in FIGS. 13D, 13E and 13F, the 3D-printed MPA with a microprojection height of 1.5 mm penetrated the deepest into the porcine skin.

Sampling of Microbiome

To compare the different sampling approaches (i.e., swab, microneedle/MPA and tape strip), Amplicon sequencing of fungal ITS regions and bacterial 16S regions were performed for species comparison, as well as for investigating species diversity and clustering.

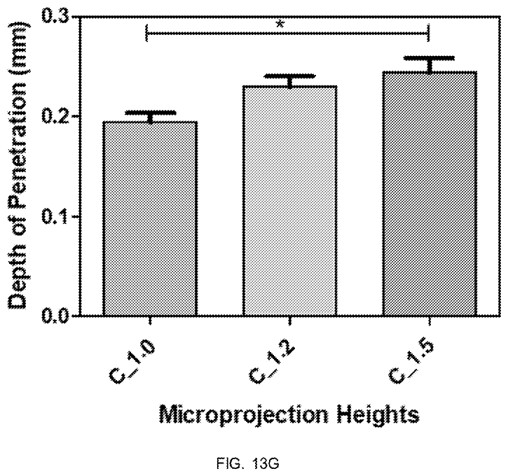

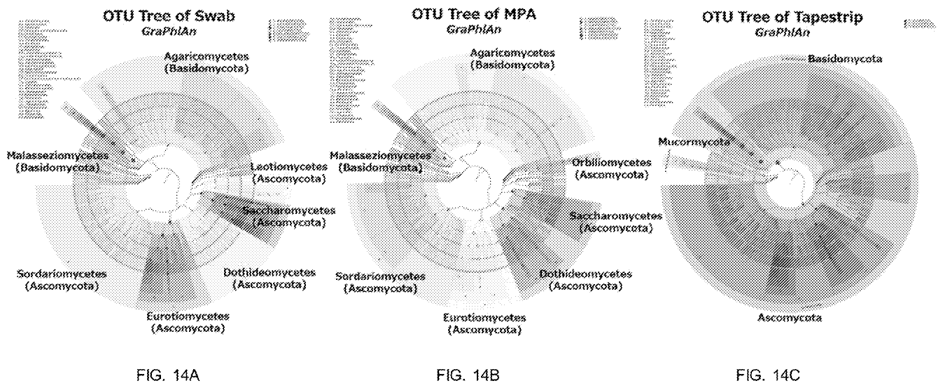

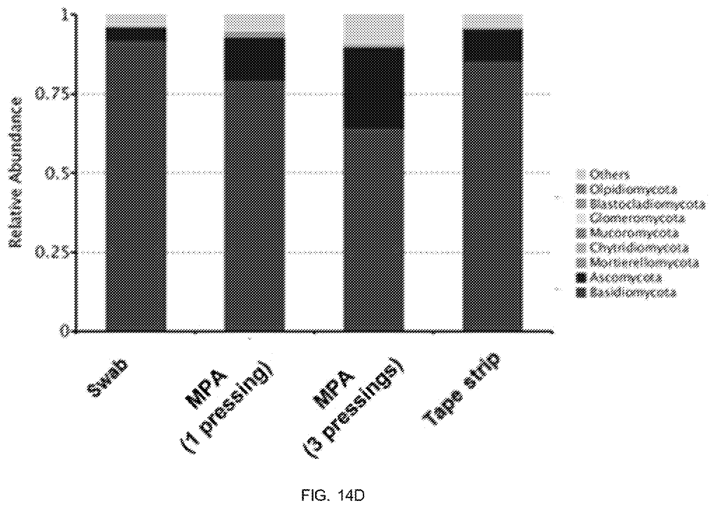

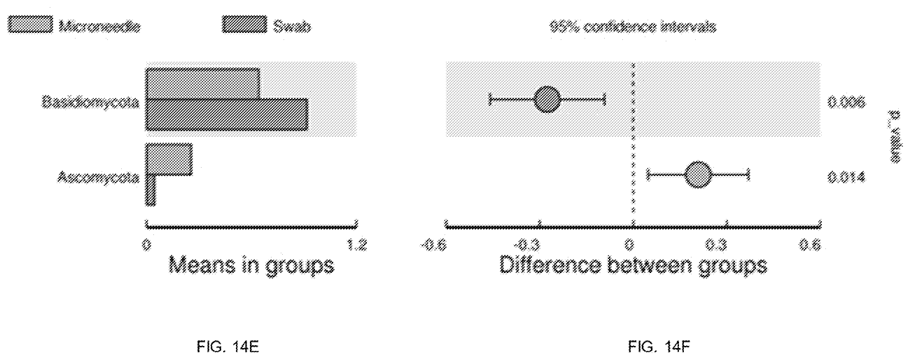

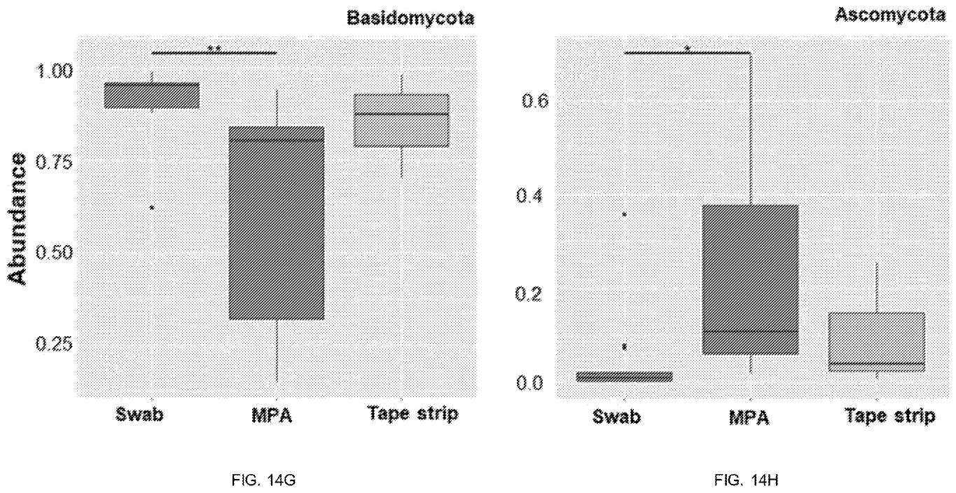

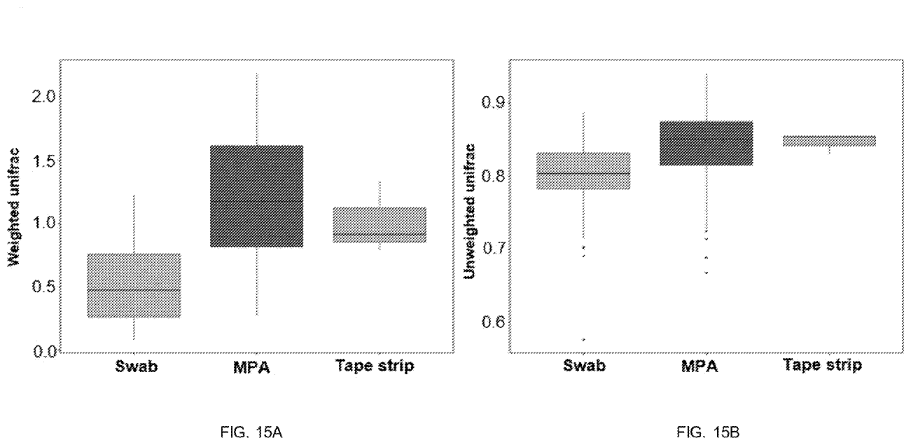

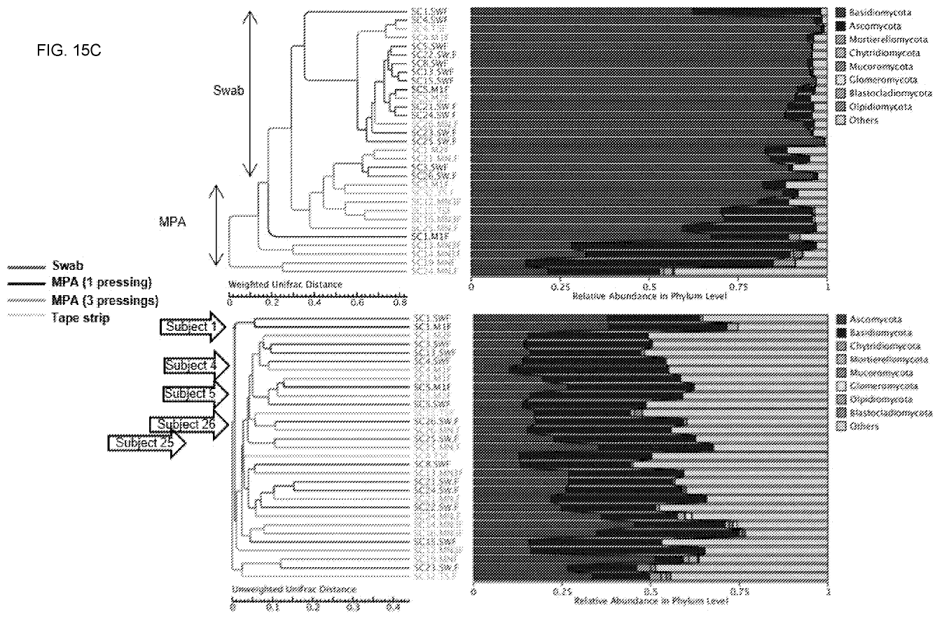

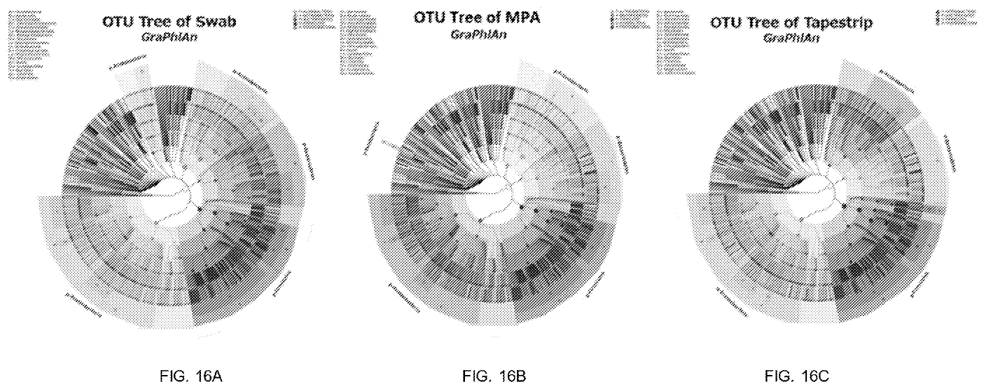

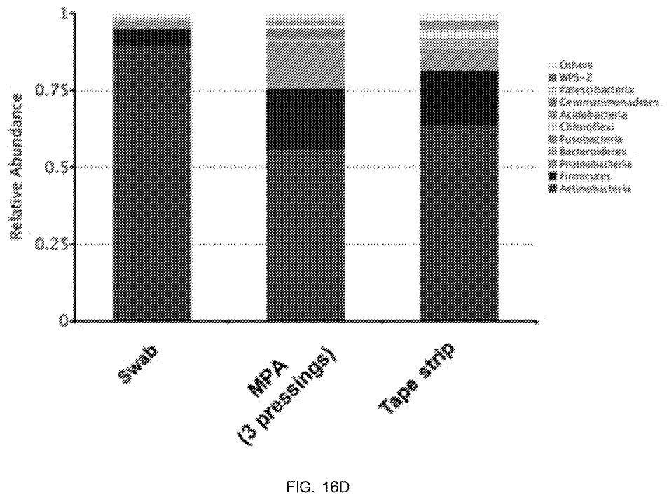

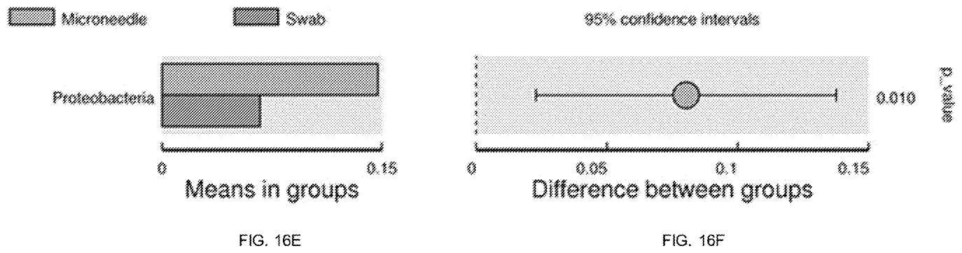

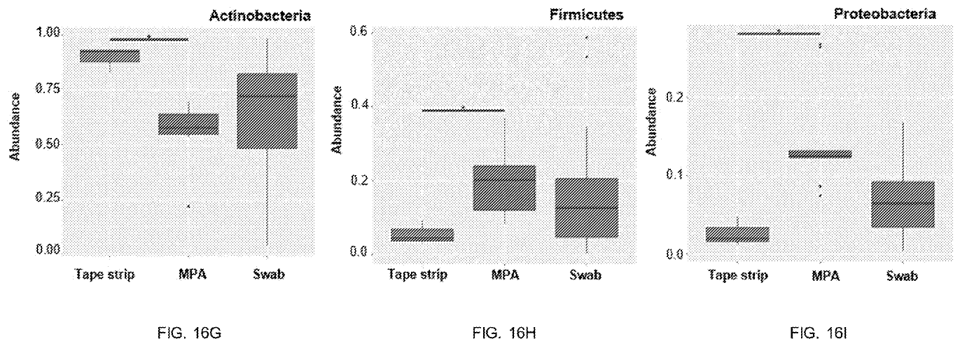

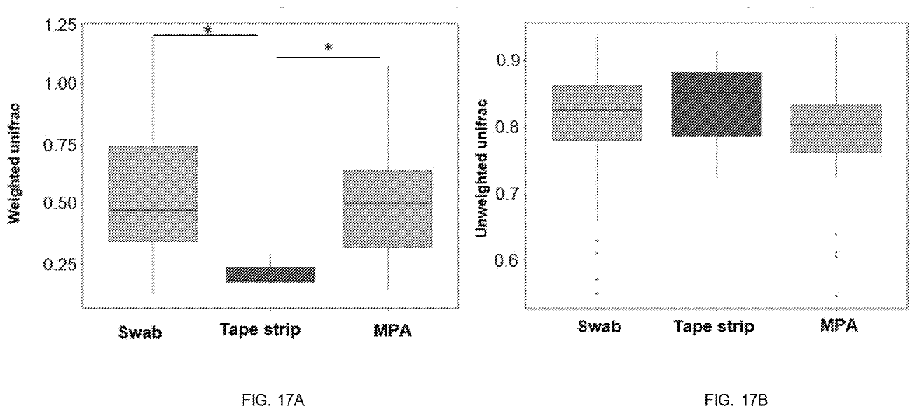

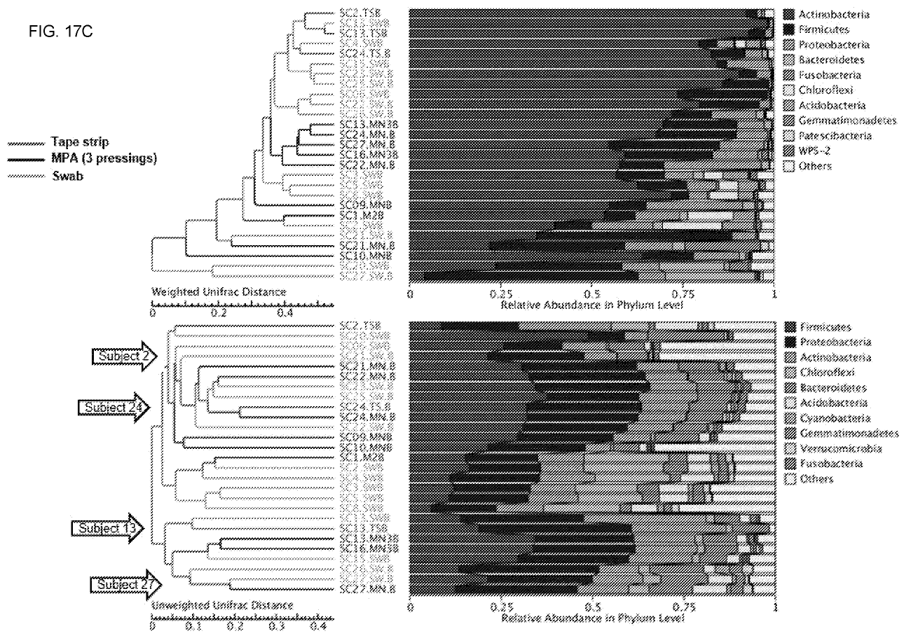

The results are shown across FIGS. 14 to 17.

Further additional data in relation to the results are shown in Table 4.

TABLE 4
Positive sampling rate for detection ITS
fungal and 16S bacterial amplicons.

| | ITS fungal positive | 16S bacterial positive |
|---|---|---|
| Swab | 13/29 (44.8%) | 15/29 (51.7%) |
| MPA | 15/29 (51.7%) | 13/29 (44.8%) |
| Tape strip | 3/29 (10.3%) | 3/29 (10.3%) |

Effect of Microprojection Heights on Microbe Extraction in an Ex Vivo Porcine Skin Model

The effect of microprojection heights in the range of 1.0 mm to 1.5 mm on microbe extraction in an ex vivo porcine skin model was evaluated.

To this end, three different types of 3D-printed MPAs were compared, namely, 3D-printed MPAs with cone-patterned microprojections of 1.0 mm, 1.2 mm and 1.5 mm (herein referred to as “C_1.0”, “C_1.2” and “C_1.5” respectively).

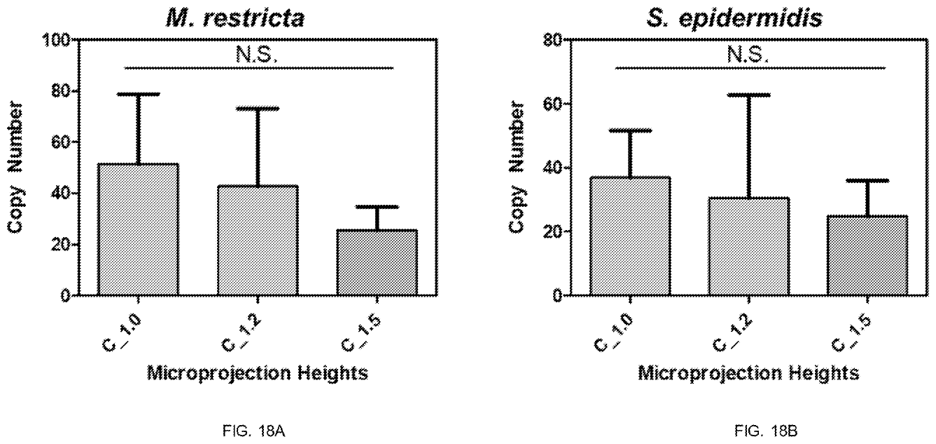

As shown by the graphs in FIGS. 18A and 18B, all MPAs with various microprojection heights (i.e., 1.0 mm, 1.2 mm and 1.5 mm) are capable of microbe extraction in an ex vivo porcine skin model.

Based on the above results, MPAs with a microprojection height of 1.2 mm for example can be used (e.g., over MPAs with microprojection height of 1.5 mm) without a reduction in efficacy of the MPAs. This may be advantageous in that MPAs with microprojection heights of 1.2 mm may cause less discomfort (e.g., to human subjects) than MPAs with microprojection heights of 1.5 mm.

Collapsible Trans-Epidermal MPA Design

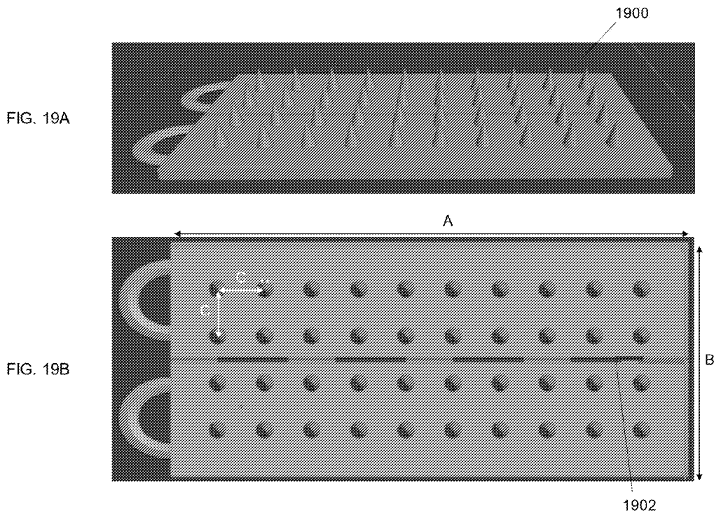

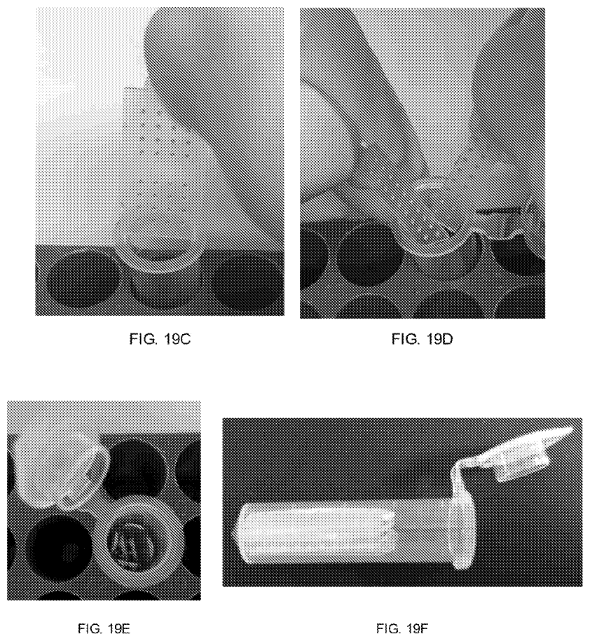

In this Example, a collapsible trans-epidermal MPA has been described. FIGS. 19A and 19B show a perspective view and a top view respectively of a computer-aided design (CAD) sketch of the collapsible trans-epidermal MPA design. As shown in FIG. 19B, the length A of the trans-epidermal MPA 1900 is 22 mm and the width B is 10 mm. The microprojections are spaced apart from one another by 2 mm (see distance C).

The trans-epidermal MPA is collapsible along the dotted line 1902 shown in FIG. 19B. A collapsible trans-epidermal MPA design may advantageously reduce the extraction buffer volume required and increase detection sensitivity. This is because, while a non-collapsible MPA design would be too wide to fit into a 2-mL Eppendorf tube (as shown in FIG. 19C), the broken halves of the collapsible MPA design may slide easily into a 2-mL Eppendorf tube (as shown in FIG. 19D). As shown in FIGS. 19E and 19F, up to three collapsed MPAs (i.e., up to six broken halves) containing sampled microbes can be fitted into one 2-mL Eppendorf tube for DNA extraction.

Efficacy of Various Sampling Tools in Extraction of Common Skin Microbes from Selected Human Samples

The efficacy of various sampling tools (i.e., swab, tape strip and MPA) in extracting skin microbes from selected human samples, namely, M. restricta, M. globosa, S. epidermidis and P. acnes was investigated.

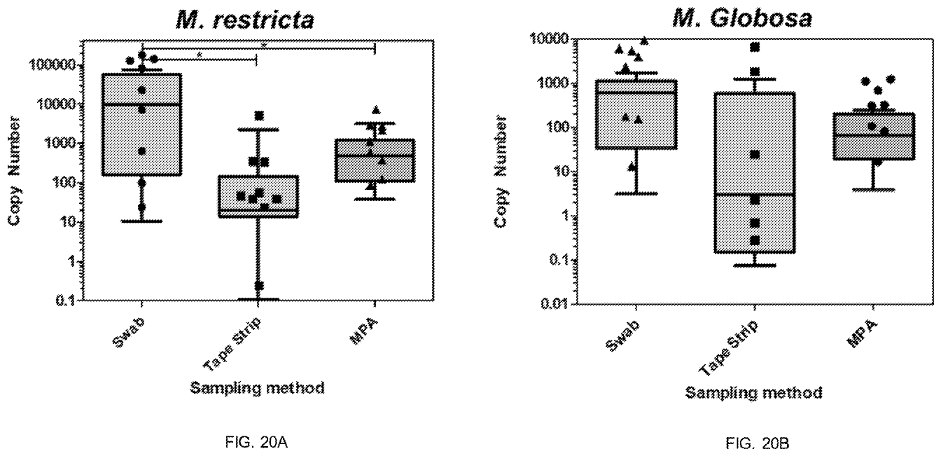

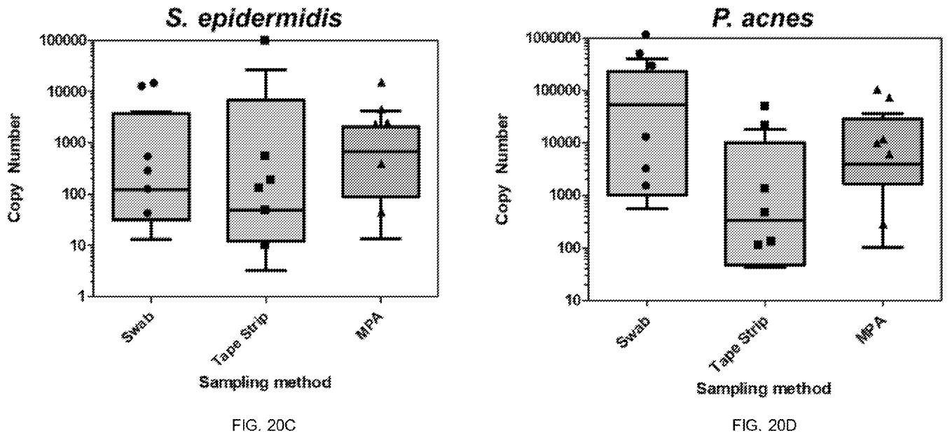

From the results shown in FIGS. 20A, 20B, 20C and 20D, the MPA performed generally comparatively well in extracting M. globosa, S. epidermidis and P. acnes as compared to the other sampling tools (i.e., swab and tape strip) (as shown in FIGS. 20B, 20C and 20D respectively). The MPA performed particularly better in extracting M. restricta as compared to the other sampling tools (i.e., swab and tape strip) (as shown in FIG. 20A).

OTU Copy Number Comparisons of Representative Fungal and Bacterial Species Derived from Amplicon Sequencing

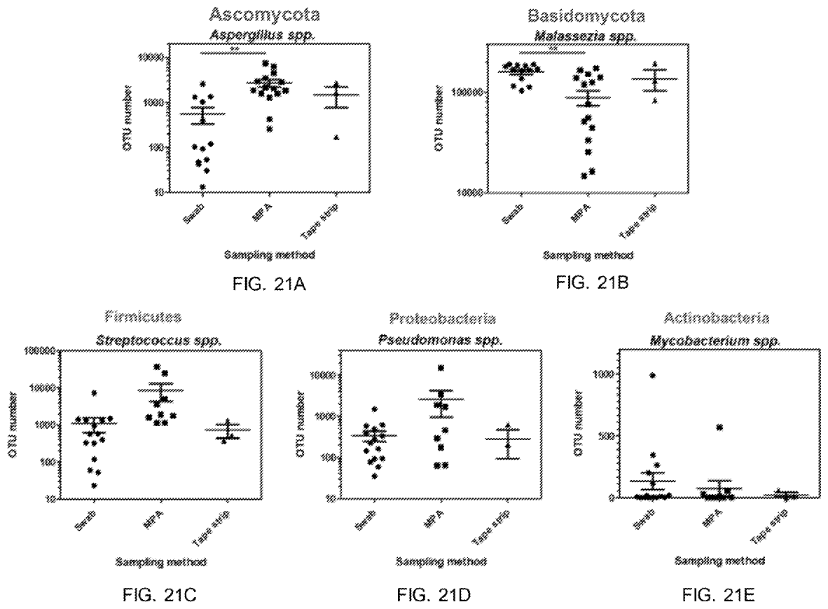

Fungal OTU copy numbers obtained for Malassezia spp. (Basidomycota) and Aspergillus spp. (Ascomycota) for different sampling methods (i.e., swab, MPA and tape strip) are shown in FIGS. 21A and 21B respectively. FIGS. 21A and 21B show that the use of MPA showed significantly improved performance in extracting/obtaining the respective fungi species as compared to swabbing.

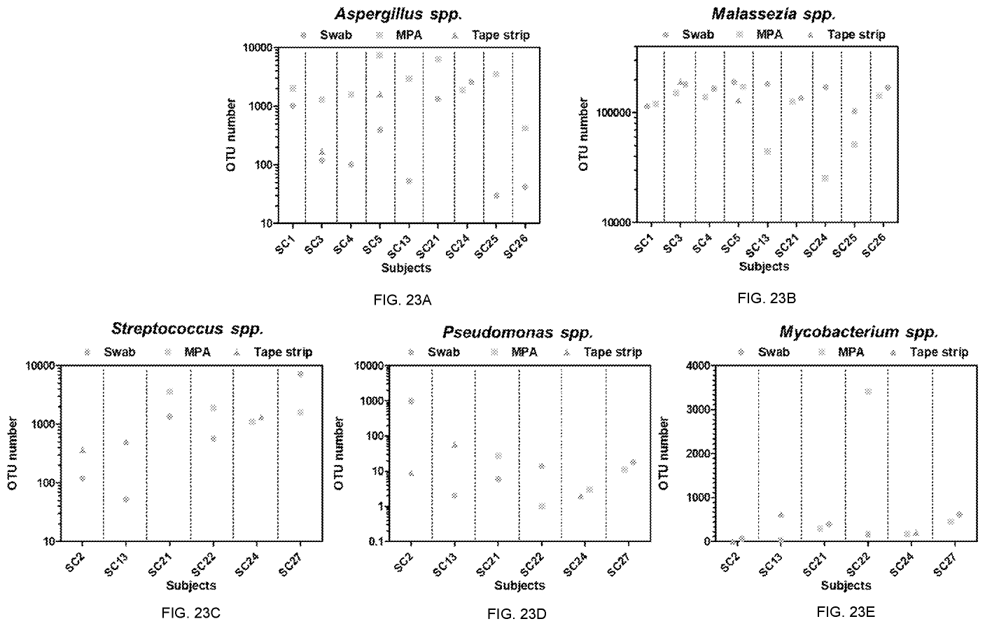

The above data reflects/correlates well with the abundance data shown in FIGS. 14G and 14H. These data support that the MPA can potentially be more clinically relevant than swabbing because the MPA can pick up pathogenic microbes such as Aspergillus better, thus making it potentially useful for clinical diagnosis applications. This is further supported by the data shown in FIGS. 23A and 23B showing fungal OTU copy numbers for Aspergillus spp. and Malassezia spp. respectively with different sampling methods (i.e., swab, MPA and tape strip) for different subjects. FIGS. 23A and 23B show that the MPA can pick up pathogenic microbes better in subjects (over swab and/or tape strip), especially with Aspergillus (see FIG. 23A).

Bacterial OTU copy numbers for Streptococcus spp. (Firmicutes), Pseudomonas spp. (Proteobacteria) and Mycobacterium spp. (Actinobacteria) for different sampling methods (i.e., swab, MPA and tape strip) are shown in FIGS. 21C, 21D and 21E respectively. FIGS. 23C, 23D and 23E show the bacterial OTU copy numbers for Streptococcus spp., Pseudomonas spp. and Mycobacterium spp. respectively with different sampling methods (i.e., swab, MPA and tape strip) for different subjects.

The Examples show that embodiments of the skin microbe sampler disclosed herein provide a unique form of skin diagnostic device which offers a disruptive technology among the conventional tools for microbe/microbiome sampling (e.g., swabbing or tape stripping) and open up a new market for such products in the future. Embodiments of the skin microbe sampler disclosed herein represent an easily translatable technology with huge commercial potential. The huge improvements in accuracy and reliability over current devices will be attractive, especially appealing to developed countries with high incidences of microbiological infections, e.g. Singapore and USA since it can still be produced at competitive pricing. Furthermore, since embodiments of the skin microbe sampler disclosed herein are minimally invasive and only for transient use (<1 min), they would be categorized under Class A medical device by the Health Sciences Authority of Singapore (HSA). This is the class of regulated devices with the lowest risk and the approval process would require simple biocompatibility and functional tests (relatively small regulatory hurdle). These validation studies can be done by sampling the skin of human volunteers.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A skin microbe sampler comprising:
a plurality of solid microneedles extending from a support surface, wherein the microneedles are dimensioned to penetrate the epidermis layer of the skin to obtain microbes residing in the skin.

2. The microbe sampler of claim 1, wherein the microneedles are configured to obtain microbes residing at a depth that is more than about 20 microns and no more than about 300 microns from the skin surface.

3. The microbe sampler of claim 1, wherein the microneedles are configured to obtain microbes residing beyond 50% of the depth of the stratum corneum of the epidermis.

4. The microbe sampler of claim 1, wherein the microneedles comprise a shape that tapers from a base to a tip.

5. The microbe sampler of claim 1, wherein the microneedles comprise a cone shape that is substantially devoid of patterning on the surface of said cone shape.

6. The microbe sampler of claim 1, wherein the microneedles have a needle height in the range of from about 1 mm to about 2.2 mm.

7. The microbe sampler of claim 1, wherein the microneedles have an aspect ratio in the range of about 0.5 to about 4.

8. The microbe sampler of claim 4, wherein the base comprises a width of from 0.5 mm to 1.5 mm.

9. The microbe sampler of claim 1, wherein the microneedles are spaced at a distance of from about 0.5 mm to about 2.0 mm from each other on the support surface.

10. The microbe sampler of claim 1, wherein the microneedles are disposed on the support at an average needle density of about 0.1 needles/mm² to about 0.6 needles/mm².

11. The microbe sampler of claim 1, wherein the microneedles are arranged in one or more 8×4 array(s).

12. The microbe sampler of claim 1, wherein the microneedles and the support surface form a unibody.

13. The microbe sampler of claim 1, wherein the microbe sampler is collapsible to reduce its original surface area for fitting into a chamber having an opening that is smaller than its original surface area.

14. The microbe sampler of claim 1, wherein the surfaces of the microneedles comprise an adhesion coating on their surfaces for enhancing adhesion of the microbes on the microneedles.

15. The microbe sampler of claim 1, wherein the microneedles are derived from 3D printing.

16. A method of sampling microbes residing in a skin, the method comprising:
contacting the microbe sampler of claim 1 with the skin to allow the microneedles to penetrate the epidermis layer of the skin;
allowing microbes residing in the skin to adhere to the microneedles that have penetrated the epidermis layer of the skin; and
removing contact of the apparatus from the skin.

17. The method of claim 16, further comprising identifying the presence of microbes on the solid microneedles after removing contact of the microbe sampler from the skin.

18. A method of making the microbe sampler of claim 1, the method comprising:
forming a plurality of solid microneedles that extends from a support surface, wherein the microneedles are dimensioned to penetrate the epidermis layer of the skin to obtain microbes residing in the skin,
wherein the forming step comprises 3D printing the solid microneedles and/or molding the solid microneedles from a negative mold of the solid microneedles.

19. The method of claim 18, further comprising coating the microneedles with an adhesion coating on their surfaces for enhancing adhesion of the microbes on the microneedles.

20. A mold for making the microbe sampler of claim 1, the mold comprising:
a negative imprint of the solid microneedles.
